(19) United States
(12) Patent Application Publication
(10) Pub. No.: US 2024/0076403 A1
(43) Pub. Date: Mar. 7, 2024

(54) HER2 ANTIBODY AND APPLICATION THEREOF

(71) Applicants: SIMCERE ZAIMING PHARMACEUTICAL CO., LTD., Haikou, Hainan (CN); JIANGSU SIMCERE PHARMACEUTICAL CO., LTD., Nanjing, Jiangsu (CN)

(72) Inventors: Xiaohui Shao, Shanghai (CN); Cuiqing Yang, Shanghai (CN); Zhuoxiao Cao, Shanghai (CN); Renhong Tang, Shanghai (CN); Jinsheng Ren, Nanjing, Jiangsu (CN)

(21) Appl. No.: 18/267,561

(22) PCT Filed: Dec. 17, 2021
(86) PCT No.: PCT/CN2021/139018

§ 371 Date: Jun. 15, 2023

(30) Foreign Application Priority Data
Dec. 18, 2020 (CN) 202011503677.9

(51) Int. Cl. C07K 16/32; A61K 45/06; A61K 47/68; A61K 49/00; A61K 51/10; C07K 14/725; C07K 16/28; G01N 33/573

(52) U.S. Cl. CPC C07K 16/32; A61K 45/06; A61K 47/6813; A61K 49/0002; A61K 51/1045; C07K 14/7051; C07K 16/2809; C07K 16/2818; C07K 16/2827; C07K 16/283; C07K 16/2863; C07K 16/2875; C07K 16/2878; G01N 33/573; C07K 2317/21; C07K 2317/24; C07K 2317/31; C07K 2317/569; G01N 2333/912

(57) ABSTRACT

An antibody or antigen-binding fragment specifically binding to HER2, a multi-specific antigen binding molecule, a chimeric antigen receptor, an immune effector cell, a nucleic acid fragment, a vector, a host cell, a pharmaceutical composition, a kit, a preparation method, and an application thereof in treatment of tumors or cancers and detection of HER2, which is of great significance for the development of HER2 antibody therapeutic drugs and detection reagents.

The present disclosure claims priority to the Chinese Patent Application No. 202011503677.9 titled “HER2 ANTIBODY AND APPLICATION THEREOF” and filed with China National Intellectual Property Administration on Dec. 18, 2020, the disclosure of which is incorporated herein by reference in its entirety.

### TECHNICAL FIELD

The present disclosure relates to the field of antibodies, in particular to an HER2 antibody and application thereof.

### BACKGROUND

HER2 is a kind of receptor tyrosine kinase encoded by HER2/neu gene and located on the surface of cell membrane, belongs to the epidermal growth factor receptor (EGFR) family. HER2 is usually correlated to signal transduction of cell growth and differentiation. HER2 has no natural ligand, but can be activated by the overexpression of a homodimer or a heterodimer of other HER family members activated by binding to ligands, thus activating the receptor tyrosine kinase; moreover, cascade reactions of the downstream signals are triggered by multiple signaling pathways such as a mitogen activated protein kinase (MAPK), a phosphatidylinositol-3-kinase-protein kinase B/Akt (PI3K-PKB/Akt), a phospholipase C-protein kinase C (PLC-PKC), and a signal transducer and activator of transcription (STAT) (Olayioye M A, Breast Cancer Res. 2001; 3(6): 385-389; Huang et al., Expert Opin Biol Ther. 2009; 9: 97-110). HER2 is expressed at a low level in very few tissues, but over-expressed in more than 30% of human tumors. Overexpression of the HER2 gene is associated with the development and progression of tumor. Moreover, HER2 gene is an important clinic treatment and prognostic indicator, and is an important target selected for a targeted tumor therapeutic drug (Cho H S and Leahy D J, Science. 2002; 297(5585): 1330-1333).

Trastuzumab (trade name: Herceptin) is a kind of recombinant humanized monoclonal anti-HER2 antibody approved by FDA of the United States in 1998. Trastuzumab targets the extracellular domain IV of HER2 protein, and blocks the ligand-independent HER2 homologous dimerization in cells over-expressing HER2 and the heterogeneous dimerization with other HER family members to a smaller extent (Cho et al., Nature. 2003; 421: 756-760; Wehrman et al., Proc Natl Acad Sci USA. 2006; 103(50): 19063-19068). Trastuzumab which is highly effective to primary invasive breast cancer patients with overexpression of HER2 has efficacy on breast cancer tumor with high expression of HER2. However, an initial respondent limited by high expression of HER2 may suffer relapse (Dinh et al., Clin Adv Hematol Oncol. 2007; 5(9): 707-717).

Pertuzumab (trade name: Perjeta) is another humanized monoclonal anti-HER2 antibody. Pertuzumab targets the domain II in which dimerization of the HER2 protein occurs, and blocks the formation of the HER2 heterodimer (Hughes et al., Mol Cancer Ther 2009; 8(7): 1885-1892). Pertuzumab has no strict demand for the high-level expression of HER2 and thus, provides more therapeutic regimens for breast cancer patients with low expression of HER2 (Franklin et al., Cancer Cell. 2004; 5(4): 317-328). The combination of Pertuzumab and Trastuzumab may enhance antineoplastic efficacy (Baselga et al., J Clin Oncol. 2010; 28: 1138-1144).

Conventional monoclonal antibodies have high molecular weight, poor tissue permeability and limited therapeutic effects. Murine monoclonal antibodies are highly immunogenic, while the affinity maturation of the engineered chimeric antibody and humanized antibody is more challenging. Fully human monoclonal antibodies have high preparation costs, long development period, low yield and other factors and thus, are limited in research, development and promotion. A class of heavy chain antibodies (HCAbs) lacking light chains was found in camel blood by Belgian scientists in 1993 for the first time. This class of antibodies contains a heavy chain variable region and two conventional CH2 and CH3 regions only, but has good structure stability and antigen-binding activity. The HCAbs and VHH domains derived thereby have advantages such as low molecular weight, flexible chemical properties, easy expression, good solubility, strong permeability, weak immunogenicity, simple humanization, ease of coupling to other molecules, which increases the diversity of drug development while making up for the defects of conventional antibodies. Therefore, there is an urgent need for the development of a novel specific VHH domain or a heavy chain antibody against HER2 effectively.

### SUMMARY

The present disclosure provides an antibody or an antigen-binding fragment specifically binding to HER2, a multispecific antigen-binding molecule, a chimeric antigen receptor, an immune effector cell, a nucleic acid fragment, a vector, a host cell, a pharmaceutical composition, a kit, a preparation method, and use thereof in the treatment of diseases and HER2 assay.

In a first aspect, the present invention relates to an antibody or an antigen-binding fragment specifically binding to HER2; the antibody or the antigen-binding fragment comprises a CDR1, a CDR2 and a CDR3, the CDR1, the CDR2 and the CDR3 comprise an HCDR1, an HCDR2 and an HCDR3 in a VHH domain set forth in any one of SEQ ID NOs: 16-33, respectively.

In some specific embodiments, the HCDR1, the HCDR2 and the HCDR3 are determined according to an IMGT numbering scheme, a Kabat numbering scheme or a Chothia numbering scheme; optionally, the HCDR1, the HCDR2 and the HCDR3 are selected from Table 9;
in some specific embodiments, the HCDR1 is selected from SEQ ID NOs: 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190 and 193;
in some specific embodiments, the HCDR2 is selected from SEQ ID NOs: 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191 and 194;
in some specific embodiments, the HCDR3 is selected from SEQ ID NOs: 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192 and 195.
preferably, the HCDR1, the HCDR2 and the HCDR3 are selected from SEQ ID NOs: 34-36, SEQ ID NOs: 37-39 and SEQ ID NOs: 40-42 according to the IMGT numbering scheme, the Kabat numbering scheme or the Chothia numbering scheme;
preferably, the HCDR1, the HCDR2 and the HCDR3 are selected from SEQ ID NOs: 43-45, SEQ ID NOs: 46-48 and SEQ ID NOs: 49-51 according to the IMGT numbering scheme, the Kabat numbering scheme or the Chothia numbering scheme;
preferably, the HCDR1, the HCDR2 and the HCDR3 are selected from SEQ ID NOs: 52-54, SEQ ID NOs: 55-57 and SEQ ID NOs: 58-60 according to the IMGT numbering scheme, the Kabat numbering scheme or the Chothia numbering scheme;
preferably, the HCDR1, the HCDR2 and the HCDR3 are selected from SEQ ID NOs: 61-63, SEQ ID NOs: 64-66 and SEQ ID NOs: 67-69 according to the IMGT numbering scheme, the Kabat numbering scheme or the Chothia numbering scheme;
preferably, the HCDR1, the HCDR2 and the HCDR3 are selected from SEQ ID NOs: 70-72, SEQ ID NOs: 73-75 and SEQ ID NOs: 76-78 according to the IMGT numbering scheme, the Kabat numbering scheme or the Chothia numbering scheme;
preferably, the HCDR1, the HCDR2 and the HCDR3 are selected from SEQ ID NOs: 79-81, SEQ ID NOs: 82-84 and SEQ ID NOs: 85-87 according to the IMGT numbering scheme, the Kabat numbering scheme or the Chothia numbering scheme;
preferably, the HCDR1, the HCDR2 and the HCDR3 are selected from SEQ ID NOs: 88-90, SEQ ID NOs: 91-93 and SEQ ID NOs: 94-96 according to the IMGT numbering scheme, the Kabat numbering scheme or the Chothia numbering scheme;
preferably, the HCDR1, the HCDR2 and the HCDR3 are selected from SEQ ID NOs: 97-99, SEQ ID NOs: 100-102 and SEQ ID NOs: 103-105 according to the IMGT numbering scheme, the Kabat numbering scheme or the Chothia numbering scheme;
preferably, the HCDR1, the HCDR2 and the HCDR3 are selected from SEQ ID NOs: 106-108, SEQ ID NOs: 109-111 and SEQ ID NOs: 112-114 according to the IMGT numbering scheme, the Kabat numbering scheme or the Chothia numbering scheme;
preferably, the HCDR1, the HCDR2 and the HCDR3 are selected from SEQ ID NOs: 115-117, SEQ ID NOs: 118-120 and SEQ ID NOs: 121-123 according to the IMGT numbering scheme, the Kabat numbering scheme or the Chothia numbering scheme;
preferably, the HCDR1, the HCDR2 and the HCDR3 are selected from SEQ ID NOs: 124-126, SEQ ID NOs: 127-129 and SEQ ID NOs: 130-132 according to the IMGT numbering scheme, the Kabat numbering scheme or the Chothia numbering scheme;
preferably, the HCDR1, the HCDR2 and the HCDR3 are selected from SEQ ID NOs: 133-135, SEQ ID NOs: 136-138 and SEQ ID NOs: 139-141 according to the IMGT numbering scheme, the Kabat numbering scheme or the Chothia numbering scheme;
preferably, the HCDR1, the HCDR2 and the HCDR3 are selected from SEQ ID NOs: 142-144, SEQ ID NOs: 145-147 and SEQ ID NOs: 148-150 according to the IMGT numbering scheme, the Kabat numbering scheme or the Chothia numbering scheme;
preferably, the HCDR1, the HCDR2 and the HCDR3 are selected from SEQ ID NOs: 151-153, SEQ ID NOs: 154-156 and SEQ ID NOs: 157-159 according to the IMGT numbering scheme, the Kabat numbering scheme or the Chothia numbering scheme;
preferably, the HCDR1, the HCDR2 and the HCDR3 are selected from SEQ ID NOs: 160-162, SEQ ID NOs: 163-165 and SEQ ID NOs: 166-168 according to the IMGT numbering scheme, the Kabat numbering scheme or the Chothia numbering scheme;
preferably, the HCDR1, the HCDR2 and the HCDR3 are selected from SEQ ID NOs: 169-171, SEQ ID NOs: 172-174 and SEQ ID NOs: 175-177 according to the IMGT numbering scheme, the Kabat numbering scheme or the Chothia numbering scheme;
preferably, the HCDR1, the HCDR2 and the HCDR3 are selected from SEQ ID NOs: 178-180, SEQ ID NOs: 181-183 and SEQ ID NOs: 184-186 according to the IMGT numbering scheme, the Kabat numbering scheme or the Chothia numbering scheme;
preferably, the HCDR1, the HCDR2 and the HCDR3 are selected from SEQ ID NOs: 187-189, SEQ ID NOs: 190-192 and SEQ ID NOs: 193-195 according to the IMGT numbering scheme, the Kabat numbering scheme or the Chothia numbering scheme.

In some specific embodiments, the CDR1, the CDR2, and/or the CDR3 comprise amino acid sequences having at most 10, 9, 8, 7, 6, 5, 4, 3, 2, or 1 mutation on the HCDR1, the HCDR2, and/or the HCDR3; the mutation may be selected from an insertion, a deletion, and/or a substitution; the substitution is preferably a substitution of conserved amino acids.

In some specific embodiments, the CDR1, the CDR2, and/or the CDR3 comprise sequences that is at least 80%, 85%, 90%, 91%, 92%, 93%, 94%, 95%, 96%, 97%, 98%, 99%, or 100% identical to the HCDR1, the HCDR2, and/or the HCDR3, respectively.

In some specific embodiments, the antibody or the antigen-binding fragment comprises a single-domain antibody comprising the CDR1, the CDR2, and the CDR3.

In some specific embodiments, the single-domain antibody comprises a sequence selected from any one of SEQ ID NOs: 16-33;
optionally, the single-domain antibody comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 16-33; the mutation is selected from an insertion, a deletion, and/or a substitution; the substitution is preferably a substitution of conserved amino acids;
optionally, the single-domain antibody comprises a sequence that is at least 80%, 85%, 90%, 91%, 92%, 93%, 94%, 95%, 96%, 97%, 98%, 99%, or 100% identical to the sequence set forth in any one of SEQ ID NOs: 16-33.

In some specific embodiments, the antibody comprises an FR region in a VHH domain set forth in any one of SEQ ID NOs: 16-33;
optionally, the antibody comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 16-33; the mutation is selected from an insertion, a deletion, and/or a substitution; the substitution is preferably a substitution of conserved amino acids;
optionally, the antibody comprises a sequence that is at least 80%, 85%, 90%, 91%, 92%, 93%, 94%, 95%, 96%, 97%, 98%, 99%, or 100% identical to the FR region in the VHH domain set forth in any one of SEQ ID NOs: 16-33.

In some specific embodiments, the antibody or the antigen-binding fragment is: (1) a chimeric antibody or a fragment thereof, (2) a humanized antibody or a fragment thereof, or (3) a fully human antibody or a fragment thereof.

In some specific embodiments, the antibody or the antigen-binding fragment comprises or does not comprise an antibody heavy chain constant region; optionally, the antibody heavy chain constant region may be selected from human, Vicugna pacos, mouse, rat, rabbit and sheep; optionally, the antibody heavy chain constant region may be selected from IgG IgM, IgA, IgE and IgD, and the IgG may be selected from IgG1, IgG2, IgG3 and IgG4; optionally, the antibody heavy chain constant region may be selected from an Fc region, a CH3 region and an intact heavy chain constant region; preferably, the antibody heavy chain constant region is a human Fc region; and preferably, the antibody or the antigen-binding fragment is a heavy chain antibody.

In some specific embodiments, the antibody or the antigen-binding fragment is further conjugated to a therapeutic agent or a tracer; preferably, the therapeutic agent is selected from a radioisotope, a chemotherapeutic agent, and an immunomodulator, and the tracer is selected from a radiocontrast medium, a paramagnetic ion, a metal, a fluorescent label, a chemiluminescent label, an ultrasound contrast agent, and a photosensitizer.

In some specific embodiments, the antibody or the antigen-binding fragment specifically binds to human HER2, monkey HER2 and/or murine HER2; preferably, the antibody or the antigen-binding fragment binds to human HER2, monkey HER2 and/or murine HER2 with a KD value of less than 1E-6 M, 1E-7 M, 2E-7 M, 3E-7 M, 4E-7 M, 5E-7 M, 6E-7 M, 8E-7 M, 9E-7 M, 1E-8 M, 2E-8 M, 3E-8 M, 4E-8 M, 5E-8 M, 6E-8 M, 8E-8 M, 9E-8 M; 1E-9 M, 2E-9 M, 3E-9 M, 4E-9 M, 5E-9 M, 6E-9 M, 8E-9 M, 9E-9 M, 1E-10 M or 1E-11 M.

In a second aspect, the present disclosure relates to an antibody or an antigen-binding fragment specifically binding to Her2; the antibody or the antigen-binding fragment comprises a CDR1, a CDR2 and a CDR3; the CDR1, the CDR2 and the CDR3 comprise an HCDR1, an HCDR2 and an HCDR3 in a VHH domain set forth in any one of SEQ ID NOs: 16-33, respectively.

In some specific embodiments, the HCDR1, the HCDR2 and the HCDR3 are determined according to an IMGT numbering scheme, a Kabat numbering scheme or a Chothia numbering scheme; optionally, the HCDR1, the HCDR2 and the HCDR3 are selected from Table 9;
optionally, the HCDR1 is selected from SEQ ID NOs: 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190 and 193;
optionally, the HCDR2 is selected from SEQ ID NOs: 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191 and 194;
optionally, the HCDR3 is selected from SEQ ID NOs: 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192 and 195;
preferably, the HCDR1, the HCDR2 and the HCDR3 are selected from SEQ ID NOs: 34-36, SEQ ID NOs: 37-39 and SEQ ID NOs: 40-42 according to the IMGT numbering scheme, the Kabat numbering scheme or the Chothia numbering scheme;
preferably, the HCDR1, the HCDR2 and the HCDR3 are selected from SEQ ID NOs: 43-45, SEQ ID NOs: 46-48 and SEQ ID NOs: 49-51 according to the IMGT numbering scheme, the Kabat numbering scheme or the Chothia numbering scheme;
preferably, the HCDR1, the HCDR2 and the HCDR3 are selected from SEQ ID NOs: 52-54, SEQ ID NOs: 55-57 and SEQ ID NOs: 58-60 according to the IMGT numbering scheme, the Kabat numbering scheme or the Chothia numbering scheme;
preferably, the HCDR1, the HCDR2 and the HCDR3 are selected from SEQ ID NOs: 61-63, SEQ ID NOs: 64-66 and SEQ ID NOs: 67-69 according to the IMGT numbering scheme, the Kabat numbering scheme or the Chothia numbering scheme;
preferably, the HCDR1, the HCDR2 and the HCDR3 are selected from SEQ ID NOs: 70-72, SEQ ID NOs: 73-75 and SEQ ID NOs: 76-78 according to the IMGT numbering scheme, the Kabat numbering scheme or the Chothia numbering scheme;
preferably, the HCDR1, the HCDR2 and the HCDR3 are selected from SEQ ID NOs: 79-81, SEQ ID NOs: 82-84 and SEQ ID NOs: 85-87 according to the IMGT numbering scheme, the Kabat numbering scheme or the Chothia numbering scheme;
preferably, the HCDR1, the HCDR2 and the HCDR3 are selected from SEQ ID NOs: 88-90, SEQ ID NOs: 91-93 and SEQ ID NOs: 94-96 according to the IMGT numbering scheme, the Kabat numbering scheme or the Chothia numbering scheme;
preferably, the HCDR1, the HCDR2 and the HCDR3 are selected from SEQ ID NOs: 97-99, SEQ ID NOs: 100-102 and SEQ ID NOs: 103-105 according to the IMGT numbering scheme, the Kabat numbering scheme or the Chothia numbering scheme;
preferably, the HCDR1, the HCDR2 and the HCDR3 are selected from SEQ ID NOs: 106-108, SEQ ID NOs: 109-111 and SEQ ID NOs: 112-114 according to the IMGT numbering scheme, the Kabat numbering scheme or the Chothia numbering scheme;
preferably, the HCDR1, the HCDR2 and the HCDR3 are selected from SEQ ID NOs: 115-117, SEQ ID NOs: 118-120 and SEQ ID NOs: 121-123 according to the IMGT numbering scheme, the Kabat numbering scheme or the Chothia numbering scheme;
preferably, the HCDR1, the HCDR2 and the HCDR3 are selected from SEQ ID NOs: 124-126, SEQ ID NOs: 127-129 and SEQ ID NOs: 130-132 according to the IMGT numbering scheme, the Kabat numbering scheme or the Chothia numbering scheme;
preferably, the HCDR1, the HCDR2 and the HCDR3 are selected from SEQ ID NOs: 133-135, SEQ ID NOs: 136-138 and SEQ ID NOs: 139-141 according to the IMGT numbering scheme, the Kabat numbering scheme or the Chothia numbering scheme;
preferably, the HCDR1, the HCDR2 and the HCDR3 are selected from SEQ ID NOs: 142-144, SEQ ID NOs: 145-147 and SEQ ID NOs: 148-150 according to the IMGT numbering scheme, the Kabat numbering scheme or the Chothia numbering scheme;
preferably, the HCDR1, the HCDR2 and the HCDR3 are selected from SEQ ID NOs: 151-153, SEQ ID NOs: 154-156 and SEQ ID NOs: 157-159 according to the IMGT numbering scheme, the Kabat numbering scheme or the Chothia numbering scheme;
preferably, the HCDR1, the HCDR2 and the HCDR3 are selected from SEQ ID NOs: 160-162, SEQ ID NOs: 163-165 and SEQ ID NOs: 166-168 according to the IMGT numbering scheme, the Kabat numbering scheme or the Chothia numbering scheme;
preferably, the HCDR1, the HCDR2 and the HCDR3 are selected from SEQ ID NOs: 169-171, SEQ ID NOs: 172-174 and SEQ ID NOs: 175-177 according to the IMGT numbering scheme, the Kabat numbering scheme or the Chothia numbering scheme;
preferably, the HCDR1, the HCDR2 and the HCDR3 are selected from SEQ ID NOs: 178-180, SEQ ID NOs: 181-183 and SEQ ID NOs: 184-186 according to the IMGT numbering scheme, the Kabat numbering scheme or the Chothia numbering scheme;
preferably, the HCDR1, the HCDR2 and the HCDR3 are selected from SEQ ID NOs: 187-189, SEQ ID NOs: 190-192 and SEQ ID NOs: 193-195 according to the IMGT numbering scheme, the Kabat numbering scheme or the Chothia numbering scheme.

In some specific embodiments, the CDR1, the CDR2, and/or the CDR3 comprise amino acid sequences having at most 10, 9, 8, 7, 6, 5, 4, 3, 2, or 1 mutation on the HCDR1, the HCDR2, and/or the HCDR3; the mutation may be selected from an insertion, a deletion, and/or a substitution; the substitution is preferably a substitution of conserved amino acids.

In some specific embodiments, the CDR1, the CDR2, and/or the CDR3 comprise sequences that is at least 80%, 85%, 90%, 91%, 92%, 93%, 94%, 95%, 96%, 97%, 98%, 99%, or 100% identical to the HCDR1, the HCDR2, and/or the HCDR3, respectively.

In some specific embodiments, the antibody or the antigen-binding fragment competitively binds to or does not competitively bind to HER2 with Trastuzumab, Pertuzumab or an FRP5 monoclonal antibody.

In some specific embodiments, the antibody or the antigen-binding fragment is further linked with an additional functional molecule; preferably, the additional functional molecule may be selected from one or more of a signal peptide, a protein tag, a cytokine, an angiogenesis inhibitor and an immune checkpoint inhibitor.

In some specific embodiments, the cytokine may be IL2, IL-6, IL-12, IL-15, IL-21, IFN or TNF-alpha; the angiogenesis inhibitor may be endostatin; and the immune checkpoint inhibitor may be SIRPα.

In a third aspect, the present disclosure further discloses a multispecific antigen-binding molecule, wherein the multispecific antigen-binding molecule comprises the antibody or the antigen-binding fragment described above, and an antigen-binding molecule binding to other antigens other than HER2 or binding to an HER2 epitope different from the antibody or the antigen-binding fragment described above; optionally, the other antigens other than HER2 may be selected from: CD3, preferably CD3E; CD16, preferably CD16A; CD137; CD258; 4-1BB; CD40; CD64; EGFR; HER1; HER3; PD-1; PD-L1; VEGF; IGF-IR (insulin-like growth factor-I receptor); phosphatidylserine (PS); c-Met; and a blood-brain barrier receptor;
preferably, the additional antigen-binding molecule is an antibody or antigen-binding fragment;
preferably, the multispecific antigen-binding molecule may be a bispecific antigen-binding molecule, a trispecific antigen-binding molecule, or a tetraspecific antigen-binding molecule; and
preferably, the multispecific antigen-binding molecule may be divalent, tetravalent or hexavalent.

In a fourth aspect, the present disclosure further discloses a chimeric antigen receptor (CAR), and the CAR comprises an extracellular antigen-binding domain, a transmembrane domain, and an intracellular signaling domain; the extracellular antigen-binding domain comprises the antibody or the antigen-binding fragment described above.

In a fifth aspect, the present disclosure further provides an immune effector cell; the immune effector cell expresses the CAR described above or comprises a nucleic acid fragment encoding the CAR described above; preferably, the immune effector cell is selected from a T cell, a natural killer (NK) cell, a natural killer T (NKT) cell, a double negative T (DNT) cell, a monocyte, a macrophage, a dendritic cell and a mastocyte; the T cell is preferably selected from a cytotoxic T cell, a regulatory T cell and a helper T cell; preferably, the immune effector cell is an autologous immune effector cell or an allogeneic immune effector cell.

In a sixth aspect, the present disclosure further discloses an isolated nucleic acid fragment; the isolated nucleic acid fragment encodes the antibody or the antigen-binding fragment, the multispecific antigen-binding molecule or the chimeric antigen receptor described above.

In a seventh aspect, the present disclosure further discloses a vector; the vector comprises the nucleic acid fragment described above.

In an eighth aspect, the present disclosure further discloses a host cell; the host cell comprises the vector described above, and preferably, the host cell is a prokaryotic cell or a eukaryotic cell, e.g., a bacterium (E. coli), a fungus (yeast), an insect cell, or a mammalian cell (CHO cell line or 293T cell line).

In a ninth aspect, the present disclosure further discloses a method for preparing the antibody or the antigen-binding fragment or the multispecific antigen-binding molecule described above; the method comprises culturing the cell described above, and isolating an antibody, an antigen-binding fragment or a multispecific antigen-binding molecule expressed by the cell.

In a tenth aspect, the present disclosure further discloses a method for preparing the immune effector cell described above; the method comprises introducing a nucleic acid fragment encoding the CAR described above into the immune effector cell, and optionally, the method further comprises initiating expression of the CAR described above by the immune effector cell.

In an eleventh aspect, the present disclosure further discloses a pharmaceutical composition; the pharmaceutical composition comprises the antibody or the antigen-binding fragment, the multispecific antigen-binding molecule, the immune effector cell, the nucleic acid fragment, or the vector described above, or a product prepared by the method described above; optionally, the pharmaceutical composition further comprises a pharmaceutically acceptable carrier, a diluent, or an adjuvant; and optionally, the pharmaceutical composition further comprises an additional antineoplastic agent.

In a twelfth aspect, the present disclosure further discloses a method for the treatment of a tumor or a cancer; the method comprises administering to a subject an effective amount of the antibody or the antigen-binding fragment, the multispecific antigen-binding molecule, the immune effector cell, the nucleic acid fragment, or the vector described above, a product prepared by the method described above, or the pharmaceutical composition described above; preferably, the tumor or the cancer is selected from a solid tumor, gastric carcinoma, gastroesophageal junction carcinoma, ovarian cancer, fallopian tube cancer, peritoneal cancer, endometrial cancer, prostatic cancer, castration resistant prostate cancer, breast cancer, HER2-positive breast cancer, sarcoma, osteosarcoma, glioblastoma multiforme, lung cancer, non-small cell lung cancer, cholangiocarcinoma, urothelium carcinoma, bladder cancer, esophageal cancer, colorectal cancer, head and neck cancers, salivary gland cancer and B-cell acute lymphocytic leukemia.

In a thirteenth aspect, the present disclosure further discloses use of the antibody or the antigen-binding fragment, the multispecific antigen-binding molecule, the immune effector cell, the nucleic acid fragment, or the vector described above, a product prepared by the method described above, or the pharmaceutical composition described above in preparing a medicament for the treatment of a tumor or a cancer; preferably, the tumor or the cancer is selected from a solid tumor, gastric carcinoma, gastroesophageal junction carcinoma, ovarian cancer, fallopian tube cancer, peritoneal cancer, endometrial cancer, prostatic cancer, castration resistant prostate cancer, breast cancer, HER2-positive breast cancer, sarcoma, osteosarcoma, glioblastoma multiforme, lung cancer, non-small cell lung cancer, cholangiocarcinoma, urothelium carcinoma, bladder cancer, esophageal cancer, colorectal cancer, head and neck cancers, salivary gland cancer and B-cell acute lymphocytic leukemia.

In a fourteenth aspect, the present disclosure further discloses the antibody or the antigen-binding fragment, the multispecific antigen-binding molecule, the immune effector cell, the nucleic acid fragment, or the vector described above, a product prepared by the method described above, or the pharmaceutical composition described above for use in treating a tumor or a cancer; preferably, the tumor or the cancer is selected from a solid tumor, gastric carcinoma, gastroesophageal junction carcinoma, ovarian cancer, fallopian tube cancer, peritoneal cancer, endometrial cancer, prostatic cancer, castration resistant prostate cancer, breast cancer, HER2-positive breast cancer, sarcoma, osteosarcoma, glioblastoma multiforme, lung cancer, non-small cell lung cancer, cholangiocarcinoma, urothelium carcinoma, bladder cancer, esophageal cancer, colorectal cancer, head and neck cancers, salivary gland cancer and B-cell acute lymphocytic leukemia.

In a fifteenth aspect, the present disclosure further provides a kit; the kit comprises the antibody or the antigen-binding fragment, the multispecific antigen-binding molecule, the immune effector cell, the nucleic acid fragment, or the vector described above, a product prepared by the method described above, or the pharmaceutical composition described above.

In a sixteenth aspect, the present disclosure further discloses a method for determining expression of HER2 in a biological sample; the method comprises contacting the biological sample with the antibody or the antigen-binding fragment described above under such conditions that the antibody or the antigen-binding fragment and HER2 can form a complex; preferably, the method further comprises determining the formation of the complex and indicating the existence or an expression level of HER2 in the sample.

In a seventeenth aspect, the present disclosure further discloses use of the antibody or the antigen-binding fragment described above in preparing an HER2 assay reagent.

### Definitions of Terms

Unless otherwise defined herein, scientific and technical terms used in correlation with the present disclosure shall have the meanings that are commonly understood by those skilled in the art. Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “including”, “comprising”, and “having” herein are used interchangeably and are intended to indicate the inclusion of a solution, implying that there may be elements other than those listed in the solution. Meanwhile, it should be understood that the descriptions “including”, “comprising”, and “having” as used herein also provides the solution of “consisting of . . . ”.

The term “and/or” as used herein includes the meanings of “and”, “or”, and “all or any other combination of elements linked by the term”.

The term “Her2” herein, also called ErbB2, NEU or CD340, includes human epidermal growth factor receptors 2 (UniProtKB ID: P04626-1) and their variants, isoforms and species homologs. The species homologs of HER2 herein include but not limited to HER2 and variants and isoforms thereof of vertebrates or mammals, for example, monkey Her2 (NCBI ID: XP 014975023.1) and mouse Her2.

The term “specific binding” herein means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), with lower KD indicating higher affinity. In the case of antibodies, high affinity generally means having a KD of about 10⁻⁶ M or less, 10⁻⁷ M or less, about 10⁻⁸ M or less, about 1×10⁹ M or less, 1×10−10 M or less, 1×10⁻¹¹ M or less, or 1×10⁻¹² M or less. KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis.

The term “antigen-binding molecule” herein is used in the broadest sense and refers to a molecule that specifically binds to an antigen. Illustratively, antigen-binding molecules include but are not limited to, antibodies or antibody mimetics. “Antibody mimetic” refers to an organic compound or a binding domain that is capable of specifically binding to an antigen, but is not structurally related to an antibody. Illustratively, antibody mimetics include but are not limited to, affibody, affitin, affilin, a designed ankyrin repeat protein (DARPin), a nucleic acid aptamer, or a Kunitz domain peptide. The term “antibody” herein is used in the broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, e.g., stabilize the three-dimensional structure of the antibody, and fully synthetic scaffolds comprising, e.g., biocompatible polymers. See, e.g., Korndorfer et al., 2003, Proteins: Structure, Function, and Bioinformatics, 53(1): 121-129 (2003); and Roque et al., Biotechnol. Prog. 20:639-654 (2004). Such scaffolds may also include non-antibody derived scaffolds, such as scaffold proteins known in the art to be useful for grafting CDRs, including but not limited to tenascin, fibronectin, peptide aptamers, and the like.

The term “antibody” herein includes a typical “four-chain antibody”, which is an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, a heavy chain constant region CH3 domain; moreover, when the full-length antibody is of IgE isoform, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are connected to each other and to the light chains through disulfide bonds to form a Y-shaped structure. The heavy chain constant regions of an immunoglobulin differ in their amino acid composition and arrangement, and thus in their antigenicity. Accordingly, “immunoglobulin” herein can be divided into five classes, or called isoforms of the immunoglobulin, namely IgM, IgD, IgG, IgA and IgE, with their corresponding heavy chains being u, 6, y, a, and E chains, respectively. The Ig of the same class can be divided into different subclasses according to the differences in the amino acid composition of the hinge regions and the number and location of disulfide bonds in the heavy chains. For example, IgG can be divided into IgG1, IgG2, IgG3, and IgG4; and IgA can be divided into IgA1 and IgA2. Light chains are divided into κ or λ, chains according to differences in the constant regions. Each of the five classes of Ig may have a κ chain or λ chain.

The term “antibody” herein also includes antibodies that do not comprise a light chain, e.g., heavy-chain antibodies (HCAbs) produced by Camelus dromedarius, Camelus bactrianus, Lama glama, Lama guanicoe, Vicugna pacos, and the like, as well as immunoglobulin new antigen receptors (Ig new antigen receptor, IgNAR) found in Chondrichthyes, e.g. e.g., shark.

The term “heavy chain antibody” herein refers to an antibody lacking a light chain of a conventional antibody. The term specifically includes but not limited to homodimeric antibodies containing a VH antigen-binding domain, CH2 and CH3 constant domains in absence of a CH1 domain.

The terms “VHH domain”, “nanobody”, and “single-domain antibody (sdAb)” herein have the same meaning and can be used interchangeably, and refer to a single-domain antibody consisting of only one heavy chain variable region constructed by cloning a variable region of a heavy chain antibody, which is the smallest antigen-binding fragment having the full function. Generally, a single-domain antibody consisting of only one heavy chain variable region is constructed by obtaining a heavy chain antibody naturally lacking a light chain and a heavy chain constant region 1 (CH1) and then cloning a variable region of an antibody heavy chain.

The “heavy chain antibody” and “single-domain antibody”, “VHH domain” and “nanobody” will be further described by reference to: Hamers-Casterman, et al., Nature. 1993; 363; 446-8; Muyldermans' review article (Reviews in Molecular Biotechnology 74: 277-302, 2001); and the following patent applications which are mentioned as general background art: WO 94/04678, WO 95/04079 and WO 96/34103; WO94/25591, WO 99/37681, WO 00/40968, WO 00/43507, WO 00/65057, WO 01/40310, WO 01/44301, EP 1134231 and WO 02/48193; WO97/49805, WO 01/21817, WO 03/035694, WO 03/054016 and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787 and WO 06/122825 as well as other prior arts mentioned in these applications.

“Antibody” herein may be derived from any animal, including but not limited to humans and non-human animals; the non-human animals may be selected from primates, mammals, rodents, and vertebrates, such as Camelidae species, Lama glama, Lama guanicoe, Vicugna pacos, sheep, rabbit, mouse, rat, or Chondrichthyes (e.g., shark).

The term “multispecific” means having at least two antigen-binding sites, each of which binds to a different epitope of the same antigen or a different epitope of a different antigen. Thus, terms such as “bispecific”, “trispecific”, and “tetraspecific” refer to the number of different epitopes to which an antibody/antigen-binding molecule can bind.

The term “valency” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

“Full-length antibody”, “complete antibody”, and “intact antibody” herein are used interchangeably and refer to an antibody having a substantially similar structure to a natural antibody.

“Antigen-binding fragment” and “antibody fragment” herein are used interchangeably and do not have the entire structure of an intact antibody, but comprise only a partial or partial variant of the intact antibody that has the ability to bind to an antigen. Illustratively, “antigen-binding fragment” or “antibody fragment” herein includes but not limited to, a Fab, an F(ab)₂, a Fab′, a Fab′-SH, an Fd, an Fv, an scFv, a diabody, and a single-domain antibody.

“Chimeric antibody” herein refers to the following antibody which has a variable sequence of immunoglobulin derived from a microbial source (e.g., rats, mice, rabbits or Vicugna pacos) and a constant region of immunoglobulin derived from different organisms (e.g., human). A method for producing the chimeric antibody is known in the art. See, for example, Morrison, 1985, Science 229 (4719): 1202-7; Oi, et al., 1986, Bio Techniques 4: 214-221; Gillies, et al., 1985 J Immunol Methods 125: 191-202; the content of which is incorporated herein by reference.

The term “humanized antibody” herein refers to a genetically engineered non-human antibody that has an amino acid sequence modified to increase homology to the sequence of a human antibody. Generally, all or part of the CDR regions of a humanized antibody are derived from a non-human antibody (donor antibody), and all or part of the non-CDR regions (e.g., variable region FRs and/or constant regions) are derived from a humanized immunoglobulin (receptor antibody). The humanized antibody generally retains or partially retains the desired properties of the donor antibody, including but not limited to, antigen specificity, affinity, reactivity, the ability to increase the activity of immune cells, the ability to enhance an immune response, or the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences. The term “variable region” herein refers to a region of the heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains of natural antibodies generally have similar structures, each of which contains four conserved framework regions (FRs) and three hypervariable regions (HVRs). See, e.g., Kindt et al., Kuby Immunology, 6th ed., W. H. Freeman and Co., p. 91 (2007). A single VH or VL domain can be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it can form precise complementarity to an epitope in a spatial structure, wherein the heavy chain variable chain CDR may be abbreviated as HCDR and the light chain variable chain CDR may be abbreviated as LCDR. The terms “framework region” or “FR region” are used interchangeably and refer to those amino acid residues of an antibody heavy chain variable region or light chain variable region, other than the CDRs. Generally, a typical antibody variable region consists of 4 FR regions and 3 CDR regions in the following order: FR1-CDR1-FR2-CDR2-FR3-CDR3-FR4.

For further description of the CDRs, reference is made to Kabat et al., J. Biol. Chem., 252: 6609-6616 (1977); Kabat et al., U.S. department of health and public services, “Sequences of proteins of immunological interest” (1991); Chothia et al., J. Mol. Biol. 196: 901-917 (1987); Al-Lazikani B. et al., J. Mol. Biol., 273: 927-948 (1997); MacCallum et al., J. Mol. Biol. 262: 732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309: 657-670 (2001). “CDR” herein may be labeled and defined in a way well known in the art, including but not limited to, the Kabat numbering scheme, the Chothia numbering scheme, or the IMGT numbering scheme; the tool sites used include but not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196: 901-917; Chothia et al., (1989) Nature 342: 878-883).

The term “IMGT numbering scheme” herein generally refers to a numbering scheme based on the international ImMunoGeneTics information system (IMGT) initiated by Lefranc et al., see Lefranc et al., Dev. Comparat. Immunol. 27: 55-77, 2003.

The term “heavy chain constant region” herein refers to the carboxyl-terminal portion of an antibody heavy chain that is not directly involved in the binding of the antibody to an antigen, but exhibits effector functions, such as interaction with an Fc receptor, which has a more conserved amino acid sequence relative to the variable domain of the antibody. The “heavy chain constant region” may be selected from: a CH1 domain, a hinge region, a CH2 domain, a CH3 domain, and a variant or fragment thereof. The “heavy chain constant region” includes a “full-length heavy chain constant region” having a structure substantially similar to that of a natural antibody constant region, and a “heavy chain constant region fragment” including only a portion of the full-length heavy chain constant region. Illustratively, a typical “full-length antibody heavy chain constant region” consists of the CH1 domain-hinge region-CH2 domain-CH3 domain. When the antibody is IgE, it further comprises a CH4 domain; and when the antibody is a heavy chain antibody, it does not include a CH1 domain. Illustratively, a typical “heavy chain constant region fragment” may be selected from Fc and CH3 domains.

The term “light chain constant region” herein refers to the carboxyl-terminal portion of an antibody light chain that is not directly involved in the binding of the antibody to an antigen. The light chain constant region may be selected from a constant κ domain and a constant λ, domain.

The term “Fc region” herein is used to define the C-terminus region of an antibody heavy chain containing at least a portion of the constant region. The “Fc region” includes Fc regions of native sequences and variant Fc regions. Illustratively, the Fc region in a human IgG heavy chain may extend from Cys226 or Pro230 to the carboxyl-terminus of the heavy chain. However, the antibody generated from a host cell may be translated and then cut; one or more, in particular one or two amino acids are cut off from the C-terminus of the heavy chain thereof. Therefore, the antibody generated by a host cell by the expression of a specific nucleic acid molecule encoding a full-length heavy chain may include a full-length heavy chain, or may include cutting variants of the full-length heavy chain. It may be such a situation when the final two C-terminal amino acids of the heavy chain are glycine (G446) and lysine (K447, the numbering way is in accordance with the Kabat EU index). Therefore, the C-terminal lysine (Lys447), or the C-terminal glycine (Gly446) and lysine (Lys447) of the Fc region may or may not be present.

Typically, the IgG Fc region comprises IgG CH2 and IgG CH3 domains; optionally, the IgG Fc region may further comprise a complete or partial hinge region, but exclude a CH1 domain. The “CH2 domain” in the human IgG Fc region generally extends from amino acid residues at the site of about 231 to amino acid residues at the site of about 340. In one embodiment, a carbohydrate chain is attached to the CH2 domain. The CH2 domain herein may be a CH2 domain of a native sequence or a variant CH2 domain. “CH3 domain” comprises the residues at the C-terminus of the CH2 domain in the Fc region (namely, from amino acid residues of IgG at the site of about 341 to amino acid residues at the site of about 447). The CH3 domain herein may be a CH3 domain of a native sequence or a variant CH3 domain (for example, the CH3 domain having a “bulge” (“knob”) introduced into one chain thereof and a “cavity” (“hole”) accordingly introduced into another chain thereof; see U.S. Pat. No. 5,821,333, the content of which is definitely incorporated herein by reference). As described herein, such kind of variant CH3 domain may be used to promote the heterodimerization of two different antibody heavy chains.

Unless otherwise specified herein, the numbering of amino acid residues in the Fc region or constant region conforms to the EU numbering scheme, also known as the EU index, as described in Kabat et al., Sequences of Proteins of Immunological Interest, 5th Ed., Public Health Service, National Institutes of Health, Bethesda, M D, 1991.

The term “conserved amino acid” herein generally refers to amino acids that belong to the same class or have similar characteristics (e.g., charge, side chain size, hydrophobicity, hydrophilicity, backbone conformation, and rigidity).

Illustratively, the following six groups are examples of amino acids that are considered to be conserved replacement of each other:
1) alanine (A), serine (S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48: 444-453; available at www.gcg.com) which has been integrated into the GAP program of the GCG software package, using the Blossom 62 matrix or PAM250 matrix and gap weight of 16, 14, 12, 10, 8, 6, or 4 and length weight of 1, 2, 3, 4, 5, or 6. For another example, the percent identity between two nucleotide acid sequences is determined with the GAP program of the GCG software package (available at www.gcg.com), using the NWSgapdna.CMP matrix and gap weight of 40, 50, 60, 70 or 80 and length weight of 1, 2, 3, 4, 5 or 6. A particularly preferred parameter set (and one that should be used unless otherwise stated) is a Blossom 62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4: 11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or correlated sequences. For example, such searches can be performed using the NBLAST and XBLAST programs (version 2.0) of Altschul et al., (1990) J Mol. Biol., 215: 403-10. BLAST nucleotide searches can be performed with the NBLAST program, with a score of 100 and a word length of 12, to obtain nucleotide sequences homologous to the nucleic acid molecule of the present disclosure. BLAST protein searches can be performed using the XBLAST program, with a score of 50 and a word length of 3, to obtain amino acid sequences homologous to the protein molecule of the present disclosure. To obtain gapped alignment results for the purpose of comparison, gapped BLAST can be used as described in Altschul et al. (1997) Nucleic Acids Res. 25: 3389-3402. When using the BLAST and gapped BLAST programs, the default parameters of the respective programs (e.g., XBLAST and NBLAST) can be used. See www.ncbi.nlm.nih.gov.

The term “chimeric antigen receptor (CAR)” herein refers to an artificial cell surface receptor engineered to be expressed on an immune effector cell and specifically binded to an antigen, which comprises at least (1) an extracellular antigen-binding domain, e.g., a heavy chain variable region or light chain variable region of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. In this context, the term “nucleic acid molecule” encompasses deoxyribonucleic acid (DNA), including, e.g., complementary DNA (cDNA) and genomic DNA; ribonucleic acid (RNA), in particular in the synthetic form of messenger RNA (mRNA), DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as a vector for direct expression of the antibodies of the present disclosure in vitro and/or in vivo, e.g., in a host or a patient. Such DNA (e.g., cDNA) or RNA (e.g., mRNA) vectors may be unmodified or modified. For example, mRNA can be chemically modified to enhance the stability of the RNA vector and/or the expression of the encoded molecule such that the mRNA can be injected into a subject to produce antibodies in vivo (see, e.g., Stadler et al., Nature Medicine 2017, published online, Jun. 12, 2017, doi: 10.1038/nm. 4356 or EP2101823B1). An “isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location. The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered.

The term “treatment” herein refers to surgical or therapeutic treatment for the purpose of preventing, slowing (reducing) the progression of an undesired physiological or pathological change, e.g., cancer or tumor. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (whether partial or complete), whether detectable or undetectable. Subjects in need of treatment include subjects already suffering from a disorder or disease as well as subjects susceptible to a disorder or disease or subjects for whom prevention of a disorder or disease is intended. When referring to terms such as slow, moderate, reduce, ameliorate, and alleviate, their meanings also include elimination, disappearance, nonoccurrence, etc.

The term “subject” herein refers to an organism that receives treatment for a particular disease or disorder described herein. Illustratively, the “subject” includes mammals, such as humans, primates (e.g., monkey), or non-primate mammals, that are being treated for a disease or disorder.

The term “effective amount” herein refers to an amount of a therapeutic agent that is effective to prevent or alleviate symptoms of a disease or the progression of the disease when administered to a cell, tissue or subject alone or in combination with another therapeutic agent. “Effective amount” also refers to an amount of a compound that is sufficient to alleviate symptoms, e.g., to treat, cure, prevent, or alleviate the associated medical disorder, or to increase the rate at which such disorder is treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

The term “EC₅₀” herein refers to the half maximum effective concentration, which includes the antibody concentration that induces a halfway response between the baseline and maximum after a specified exposure time. EC₅₀ essentially represents the antibody concentration by which 50% of its maximum effect is observed, and can be measured by methods known in the art.

### BRIEF DESCRIPTION OF THE DRAWING

FIG. 1A shows assay results for binding reactions of control antibodies with a human HER2-His protein by ELISA;

FIG. 1B shows assay results for binding reactions of control antibodies with a monkey HER2-His protein by ELISA;

FIGS. 2A-2D show assay results for expression level of HER2 in SK-BR-3 cells via control antibodies by FACS;

FIGS. 3A-3F show assay results for screening for CHO-K1 cells transfected with a human HER2 protein by FACS;

FIG. 4 shows assay results for expression level of HER2 in HEK293T-monkey HER2 cells via a Tab048 antibody by FACS;

FIG. 5A shows assay results for serum antibody titer of Vicugna pacos after being immunized by a human HER2 protein by ELISA;

FIG. 5B shows assay results for serum antibody titer of Vicugna pacos after being immunized by a human HER2 protein by FACS;

FIGS. 6A-6C show assay results for binding reactions of VHH-hFc with a human HER2-His protein by ELISA;

FIGS. 7A-7C show assay results for binding reactions of VHH-hFc with human HER2-CHO-K1 cells by ELISA;

FIGS. 8A-8C show assay results for binding reactions of VHH-hFc with SK-BR-3 tumor cells by ELISA;

FIGS. 9A-9C show assay results for binding reactions of VHH-hFc with a monkey HER2-His protein by ELISA;

FIGS. 10A-10C show assay results for binding reactions of VHH-hFc with a murine HER2-His protein by ELISA;

FIGS. 11A-11C show assay results for binding reactions of VHH-hFc with HEK293T-monkey HER2 cells by ELISA;

FIGS. 12A-12B show assay results for affinity of VHH-hFc with human HER2 by SPR;

FIGS. 13A-13B show assay results for affinity of VHH-hFc with monkey HER2 by SPR;

FIG. 14 shows assay results for affinity of VHH-hFc with murine HER2 by SPR;

FIG. 15 shows an inhibition ratio between VHH-hFc antibodies detected by competitive ELISA; and

FIG. 16 shows classification of epitopes of VHH-hFc.

### DETAILED DESCRIPTION

The present disclosure is further described below with reference to specific examples; the advantages and features of the present disclosure will become more apparent with the description. Experimental procedures without specified conditions in the examples are conducted according to conventional conditions or conditions recommended by the manufacturer. Reagents or instruments without specified manufacturers used herein are conventional products that are commercially available.

The examples herein are exemplary only, and do not limit the scope of the present disclosure in any way. It will be understood by those skilled in the art that various modifications or substitutions may be made to the technical solutions of the present disclosure in form and details without departing from the spirit and scope of the present disclosure, and that these modifications and substitutions shall fall within the protection scope of the present disclosure.

### Example 1. Preparation of Control Antibodies, Identification of Endogenous Cells and Preparation of Over-Expression Cell Strain

(A) Preparation of Control Antibodies

VH and VL sequences of monoclonal antibodies FRP5, 4D5 (Trastuzumab) and 2C4 (Pertuzumab) identifying human HER2 were recombined into human IgG1 CH and CL expression vectors (the expression vectors were purchased from Biointron and the recombination steps were completed by Biointron as well), to obtain recombinant plasmids (experimental principles and steps of the above plasmid recombination are referring to Molecular Cloning: A Laboratory Manual (3rd Edition), (U.S.) Sambrook. J. et al.). VH and VL sequences of the monoclonal antibody FRP5 were linked with each other via 3 GGGGS linkers and then recombined into the expression vectors of human IgG1 Fc to obtain recombinant plasmids, then subjected to sequencing and validation. High-purity recombinant plasmids, with a mass of 500 μg above, were extracted at a mediate dose via an alkaline lysis kit (purchased from QIAGEN), and filtered through a 0.22 μm membrane filter (purchased from Millopore) for transfection. The above plasmid construction, antibody expression and purification work were completed by Biointron. 293F cells (purchased from Invitrogen) were cultured in a 293 medium (medium A, purchased from Biointron). A shaker was set at 37° C., 120 RPM and 8% CO₂ (v/v). 2 mL of the medium A and 120 μl of 1 μg/mL PEI (purchased from Biointron) were mixed to obtain medium B. 2 mL of the medium A and of 30 μg of the recombinant plasmid were mixed to obtain medium C. 5 minutes later, the medium B and medium C were combined and mixed, and left to stand for 15 min to obtain a mixed solution D. 4 mL of the mixed solution D was slowly added to 60 mL of the 293 medium containing 293F cells until the 293F cells had a density of 1.5×10⁶/mL, and then placed into the shaker for culture, where the 293 medium was oscillated while the mixed solution was added to avoid the excessive aggregation of PEI. After the cells were cultured for consecutive 5 days, supernatant was collected and centrifuged for 5 min at 8000 rpm, then cells were discarded and the supernatant was purified.

Continuously produced endotoxin-free chromatography columns and protein A packings were treated with 0.1 M NaOH for 30 min or washed with 0.5M NaOH in 5 column volumes; column packings and chromatography columns not used for a long time were soaked with at least 1 M NaOH for 1 h, and washed until neutral with endotoxin-free water; the column packings were washed with 1% Triton X100 in 10 times of column volume. The column was balanced with PBS in column volumes, and the centrifuged cell supernatant was loaded on the column, and flow-through was collected if necessary. The column was washed with PBS in 5 column volumes after the centrifuged cell supernatant was loaded on the column. The column was eluted with a citrate buffer solution (pH 3.4) in 5 times of column volume, and eluent was collected and neutralized with 1/10 volume of 1 M Tris (pH 8.0). Antibodies were dialyzed in 1×PBS over the night after harvesting to avoid contamination by endotoxin. After the completion of dialysis, concentrations were assayed by Nanodrop; purity of the antibodies was assayed by HPLC-SEC; the content of endotoxin in the antibodies was assayed by an endotoxin assay kit (purchased from Charles River Laboratories International, Inc.).

Antibodies in the form of FRP5 human IgG1, 4D5 human IgG1, 2C4 human IgG1 and FRP5 ScFv-human IgG1 Fc (hFc), respectively were named as Tab048, Tab049, Tab050 and Tab094. Detailed sequence information is shown in Table 1.

TABLE 1
Sequence information of the control antibodies

| Sequence name | Amino acid sequence |
|---|---|
| Tab048 heavy | QVQLQQSGPELKKPGETVKISCKASGYPFTNYGMNWVKQAPGQGLKWMGWI |
| chain (FRP5 VH is | NTSTGESTFADDFKGRFDFSLETSANTAYLQINNLKSEDMATYFCARWEVYHG |
| underlined) | YVPYWGQGTTVTVSSASTKGPSVFPLAPSSKSTSGGTAALGCLVKDYFPEPVTV |
| (SEQ ID NO: 1) | SWNSGALTSGVHTFPAVLQSSGLYSLSSVVTVPSSSLGTQTYICNVNHKPSNTK |
| | VDKKVEPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNV |
| | FSCSVMHEALHNHYTQKSLSLSPGK |
| Tab048 light chain | DIQLTQSHKFLSTSVGDRVSITCKASQDVYNAVAWYQQKPGQSPKLLIYSASSR |
| (FRP5 VL is | YTGVPSRFTGSGSGPDFTFTISSVQAEDLAVYFCQQHFRTPFTFGSGTKLEIKRTV |
| underlined) | AAPSVFIFPPSDEQLKSGTASVVCLLNNFYPREAKVQWKVDNALQSGNSQESV |
| (SEQ ID NO: 2) | TEQDSKDSTYSLSSTLTLSKADYEKHKVYACEVTHQGLSSPVTKSFNRGEC |
| Tab049 heavy | EVQLVESGGGLVQPGGSLRLSCAASGFNIKDTYIHWVRQAPGKGLEWVARIYP |
| chain (4D5 VH is | TNGYTRYADSVKGRFTISADTSKNTAYLQMNSLRAEDTAVYYCSRWGGDGFYA |
| underlined) | MDYWGQGTLVTVSSASTKGPSVFPLAPSSKSTSGGTAALGCLVKDYFPEPVTV |
| (SEQ ID NO: 3) | SWNSGALTSGVHTFPAVLQSSGLYSLSSVVTVPSSSLGTQTYICNVNHKPSNTK |
| | VDKKVEPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNV |
| | FSCSVMHEALHNHYTQKSLSLSPGK |
| Tab049 light chain | DIQMTQSPSSLSASVGDRVTITCRASQDVNTAVAWYQQKPGKAPKLLIYSASFL |
| (4D5 VL is | YSGVPSRFSGSRSGTDFTLTISSLQPEDFATYYCQQHYTTPPTFGQGTKVEIKRTV |
| underlined) | AAPSVFIFPPSDEQLKSGTASVVCLLNNFYPREAKVQWKVDNALQSGNSQESV |
| (SEQ ID NO: 4) | TEQDSKDSTYSLSSTLTLSKADYEKHKVYACEVTHQGLSSPVTKSFNRGEC |
| Tab050 heavy | EVQLVESGGGLVQPGGSLRLSCAASGFTFTDYTMDWVRQAPGKGLEWVADVN |
| chain (2C4 VH is | PNSGGSIYNQRFKGRFTLSVDRSKNTLYLQMNSLRAEDTAVYYCARNLGPSFYF |
| underlined) | DYWGQGTLVTVSSASTKGPSVFPLAPSSKSTSGGTAALGCLVKDYFPEPVTVS |
| (SEQ ID NO: 5) | WNSGALTSGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNG |
| | KEYKCKVSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRWQQGNVFS |
| | CSVMHEALHNHYTQKSLSLSPGK |
| Tab050 light chain | DIQMTQSPSSLSASVGDRVTITCKASQDVSIGVAWYQQKPGKAPKLLIYSASYR |
| (2C4 VL is | YTGVPSRFSGSGSGTDFTLTISSLQPEDFATYYCQQYYIYPYTFGQGTKVEIKRT |
| underlined) | VAAPSVFIFPPSDEQLKSGTASVVCLLNNFYPREAKVQWKVDNALQSGNSQES |
| (SEQ ID NO: 6) | VTEQDSKDSTYSLSSTLTLSKADYEKHKVYACEVTHQGLSSPVTKSFNRGEC |
| Tab94 single | QVQLQQSGPELKKPGETVKISCKASGYPFTNYGMNWVKQAPGQGLKWMGWI |
| strand (FRP5 scFv | NTSTGESTFADDFKGRFDFSLETSANTAYLQINNLKSEDMATYFCARWEVYHG |
| is underlined) | YVPYWGQGTTVTVSSGGGGSGGGGSGGGGSDIQLTQSHKFLSTSVGDRVSITC |
| (SEQ ID NO: 7) | KASQDVYNAVAWYQQKPGQSPKLLIYSASSRYTGVPSRFTGSGSGPDFTFTISS |
| | VQAEDLAVYFCQQHFRTPFTFGSGTKLEIKEPKSADKTHTCPPCPAPELLGGPSV |
| | FLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| hFc | EPKSADKTHTCPPCPAPELLGGPSVFLFPPKPKDTLMISRTPEVTCVVVDVSHED |
| (SEQ ID NO: 8) | PEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVM |
| | HEALHNHYTQKSLSLSPGK |
| CH (human IgG) | ASTKGPSVFPLAPSSKSTSGGTAALGCLVKDYFPEPVTVSWNSGALTSGVHTFP |
| (SEQ ID NO: 9) | AVLQSSGLYSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAP |
| | IEKTISKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALHNHYT |
| | QKSLSLSPGK |
| CL (human IgG) | RTVAAPSVFIFPPSDEQLKSGTASVVCLLNNFYPREAKVQWKVDNALQSGNSQ |
| (SEQ ID NO: 10) | ESVTEQDSKDSTYSLSSTLTLSKADYEKHKVYACEVTHQGLSSPVTKSFNRGEC |

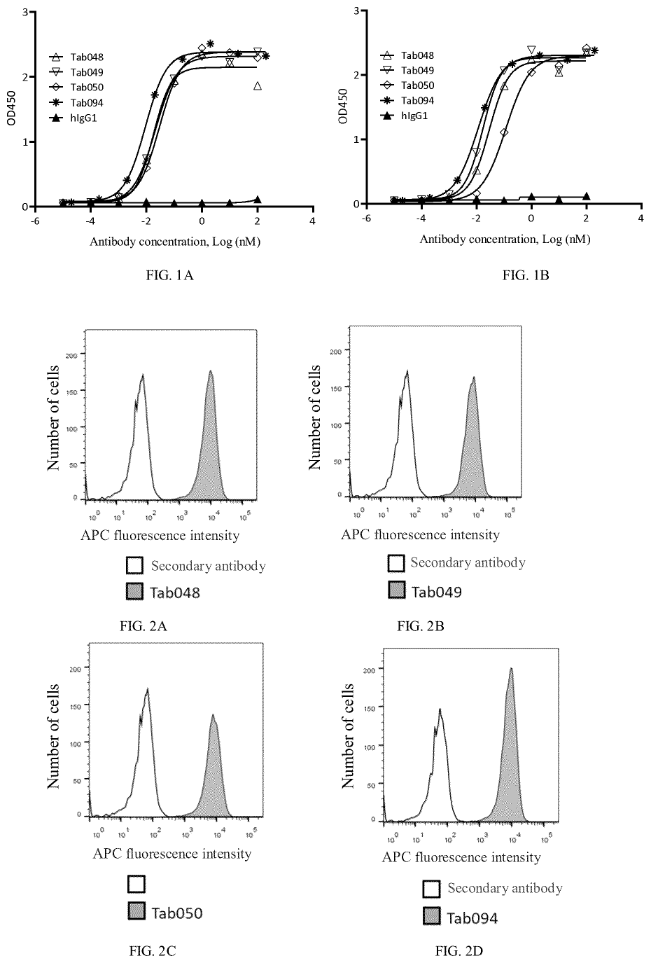

The binding activity of control antibodies to a human HER2-His protein (purchased from Acro, Cat. No. HE2-H5225) and a monkey HER2-His protein (purchased from Sino Biological, Cat. No. 90295-C08H) was assayed by ELISA. Detailed assay results are shown in Table 2-3 and FIGS. 1A-1B, of which the IgG subtype control is hIgG1 and data in the tables are values of OD₄₅₀. The results show that antibodies Tab048, Tab049, Tab050 and Tab094 had good binding activity to human and monkey HER2 proteins.

TABLE 2
Assay results for binding reactions of control antibodies with human HER2-his protein by
ELISA

| Antibody | | | | | Antibody | |
|---|---|---|---|---|---|---|
| concentration | | | | | concentration | |
| (nM) | Tab048 | Tab049 | Tab050 | hIgG1 | (nM) | Tab094 |
| 100 | 1.87 | 2.38 | 2.30 | 0.11 | 200 | 2.32 |
| 10 | 2.22 | 2.22 | 2.37 | 0.06 | 20 | 2.36 |
| 1 | 2.36 | 2.31 | 2.45 | 0.06 | 2 | 2.51 |
| 0.1 | 1.95 | 1.97 | 1.90 | 0.05 | 0.2 | 2.27 |
| 0.01 | 0.72 | 0.74 | 0.59 | 0.07 | 0.02 | 1.72 |
| 0.001 | 0.13 | 0.14 | 0.17 | 0.06 | 0.002 | 0.40 |
| 0.0001 | 0.06 | 0.06 | 0.06 | 0.06 | 0.0002 | 0.12 |
| 0.00001 | 0.05 | 0.05 | 0.06 | 0.05 | 0.00002 | 0.07 |

TABLE 3
Assay results for binding reactions of control antibodies with monkey HER2-his protein by
ELISA

| Antibody | | | | | Antibody | |
|---|---|---|---|---|---|---|
| concentration | | | | | concentration | |
| (nM) | Tab048 | Tab049 | Tab050 | hIgG1 | (nM) | Tab094 |
| 100 | 2.35 | 2.35 | 2.42 | 0.12 | 200 | 2.38 |
| 10 | 2.04 | 2.07 | 2.15 | 0.07 | 20 | 2.23 |
| 1 | 2.24 | 2.39 | 2.04 | 0.11 | 2 | 2.30 |
| 0.1 | 1.83 | 2.07 | 1.11 | 0.05 | 0.2 | 2.17 |
| 0.01 | 0.52 | 0.79 | 0.16 | 0.08 | 0.02 | 1.49 |
| 0.001 | 0.11 | 0.14 | 0.06 | 0.05 | 0.002 | 0.36 |
| 0.0001 | 0.05 | 0.06 | 0.06 | 0.06 | 0.0002 | 0.09 |
| 0.00001 | 0.05 | 0.05 | 0.05 | 0.05 | 0.00002 | 0.05 |

(B) Identification of the cell strain endogenously expressing HER2 protein SK-BR-3 cells were expanded to a logarithmic growth phase in a T-75 cell culture flask, the culture supernatant was discarded by centrifugation, and the cell pellet was washed twice with PBS. Assay and analysis were performed by FACS (FACS Canto™, purchased from BD Biosciences) using the antibodies Tab048, Tab049, Tab050 and Tab094 as primary antibodies and an APC-labeled secondary antibody (purchased from Biolegend, Cat. No. 409306). The results are shown in Table 4 and FIGS. 2A-2D, which indicates that SK-BR-3 cells may bind to Tab048, Tab049, Tab050 and Tab094.

TABLE 4
Assay results for HER2 expression level of SK-BR-3 cells by
FACS

| | | Mean fluorescence intensity of cells | | | | |
|---|---|---|---|---|---|---|
| | Endogenous- | Secondary | | | | |
| | expression | antibody | | | | |
| No. | cell line | control | Tab048 | Tab049 | Tab050 | Tab094 |
| 1 | SK-BR-3 | 58 | 8852 | 7730 | 7849 | 8425 |

(C) Preparation the CHO-K1 Recombinant Cell Strain Expressing Human HER2 Protein

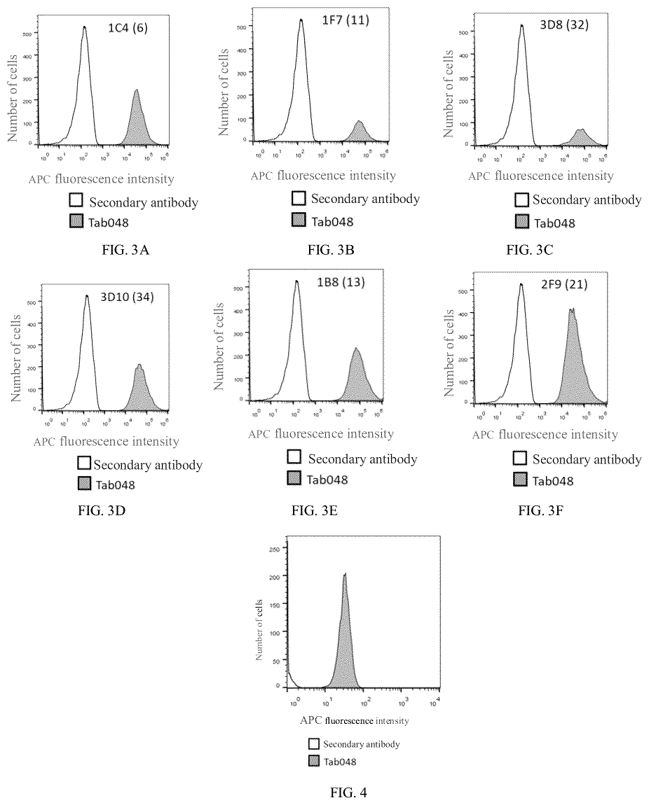

A nucleotide sequence encoding a full-length amino acid sequence of human HER2 protein (UniProtKB ID: P04626-1, website: https://www.uniprot.org/uniprot/P04626, isoform 1) was cloned into a pcDNA3.1 vector (purchased from Clontech), and a plasmid was prepared. After transfection of the CHO-K1 cell line (purchased from National Collection of Authenticated Cell Cultures of Chinese Academy of Sciences) with the plasmid (Lipofectamine® 3000 Transfection Kit, purchased from Invitrogen, Cat. No. L3000-015), the cells were selectively cultured for 2 weeks in a DMEM/F12 medium containing 10 μg/mL puromycin and 10% (w/w) fetal bovine serum (FBS); the expression level was assayed with a flow cytometer. Monoclones with high expression were selected by a limiting dilution method, namely, the cells were seeded into a 96-well plate after cell density was adjusted to 0.5/well, then the plate was placed and cultured at 37° C., 5% (v/v) CO₂, and about 2 weeks later, a portion of monoclonal wells were selected and amplified. The amplified clones were screened by flow cytometry. Monoclonal cell lines with better growth and higher fluorescence intensity were selected for further expansion and cryopreserved in liquid nitrogen. The specific results for the selection are shown in Table 5 and FIGS. 3A-3F, in which the sample incubated with the secondary antibody only served as a control. The assay results in Table 5 indicate that a series of CHO-K1 monoclonal cell lines with positive expression of human HER2 have been prepared. In FIGS. 3A-3F, x-coordinate denotes the fluorescence intensity of cells, and y-coordinate denotes the number of cells. Results indicate that 1B8(13), 3D8(32), 1F7(11), 3D10(34), 2F9(21) and 1C4(6) were cell strains with high-level expression of human HER2, and 1F7(11) clones were used for the subsequent screening.

TABLE 5
Assay results for the CHO-K1 recombinant cell
line expressing human HER2 protein by FACS

| | Clone No. of stably | | Mean fluorescence intensity of cells | |
|---|---|---|---|---|
| No. | transfected cell line | | Secondary antibody control | Tab048 |
| 1 | 1B8 | (13) | 147 | 112133 |
| 2 | 3D8 | (32) | 147 | 99341 |
| 3 | 1F7 | (11) | 147 | 70652 |
| 4 | 3D10 | (34) | 147 | 70810 |
| 5 | 2F9 | (21) | 147 | 58459 |
| 6 | 1C4 | (6) | 147 | 48505 |

(D) Preparation of the Recombinant HEK293T Cell Strain Expressing Monkey HER2 Protein

A nucleotide sequence encoding a full-length amino acid sequence of monkey HER2 (NCBI ID: XP_014975023.1, website: https://www.ncbi.nlm.nih.gov/protein/XP_014975023.1/) was cloned into a pcDNA3.1 vector (purchased from Thermofisher scientific), and a plasmid was prepared. After plasmid transfection of the HEK293T cell line with FuGENE® HD (Promega, Cat. No. E2311), the cells were selectively cultured in a DMEM medium containing 5 μg/mL puromycin and 10% (w/w) FBS for 2 weeks, subcloned in a 96-well culture plate by a limiting dilution method, and cultured at 37° C. with 5% (v/v) CO₂. About 2 weeks later, some of the polyclonal wells were selected and amplified in a 6-well plate. The amplified clones were assayed and analyzed by an FACS flow cytometer using a Tab048 antibody, and cell strains with better growth and higher fluorescence intensity were selected for further expansion and cryopreserved in liquid nitrogen. Results of the expression level are shown in FIG. 4, indicating that the HEK293T-monkey-HER2 had a single positive cell peak after puromycin pressurization screening, and thus may be used for detecting the cross-activity of the antibodies.

### Example 2. Preparation of Single-Domain Antibody VHH Against HER2

(A) Vicugna pacos Immunization and Serum Titer Assay

Two Vicugna pacos (Alpaca, No. NB146 and No. NB147) were immunized with human HER2 (Thr23-Thr652)-His protein (purchased from Acro, Cat. No. HE2-H82E2). The human HER2 protein was emulsified with a Freund's complete adjuvant and then injected subcutaneously with multipoints at the primary immunization, namely, each Vicugna pacos was injected with 500 μg of the human HER2-His protein. The human HER2 protein was emulsified with a Freund's incomplete adjuvant and then injected subcutaneously with multipoints at the booster immunization, namely, each Vicugna pacos was injected with 250 μg of the human HER2-His protein. The primary immunization and the first booster immunization were performed at an interval of 3 weeks, and the remaining booster immunizations were performed at intervals of 3 weeks. Blood sampling was performed 1 week later after each booster immunization; titer and specificity of the human HER2-His antibody in serum were assayed by ELISA and FACS. Results are shown in FIGS. 5A-5B and Tables 6-7. The results indicate that serum from the Vicugna pacos immunized by the human HER2-His protein bound to the immunogen to different extent, and presented antigen-antibody reactions with the maximum dilution of ELISA being around 24,300. The blank control is 1% (w/w) BSA, and batches refer to the Vicugna pacos serum on the 7th day after the third (TB2) and the fourth (TB3) immunizations; data in the table are values of OD_{450 nm}.

TABLE 6
Serum antibody titer of Vicugna pacos after being immunized
with a human HER2-His protein assayed by ELISA

Batch

| | NB146 | NB146 | NB147 | NB147 |
|---|---|---|---|---|
| Serum dilution | (TB2) | (TB3) | (TB2) | (TB3) |
| 1:100 | 2.59 | 2.17 | 2.21 | 2.33 |
| 1:300 | 2.44 | 2.21 | 2.14 | 2.24 |
| 1:900 | 2.75 | 2.17 | 2.11 | 2.23 |
| 1:2700 | 2.41 | 1.89 | 1.79 | 1.89 |
| 1:8100 | 1.35 | 1.18 | 1.08 | 1.10 |
| 1:24300 | 0.63 | 0.43 | 0.42 | 0.42 |
| 1:72900 | 0.21 | 0.15 | 0.15 | 0.15 |
| 1:218700 | 0.11 | 0.08 | 0.07 | 0.08 |
| 1:656100 | 0.08 | 0.06 | 0.06 | 0.06 |
| 1:1968300 | 0.07 | 0.06 | 0.06 | 0.06 |
| 1:5904900 | 0.08 | 0.06 | 0.06 | 0.06 |
| Blank control | 0.08 | 0.07 | 0.07 | 0.06 |

TABLE 7
Serum antibody titer of Vicugna pacos after being
immunized with a human HER2 protein assayed by FACS
Mean fluorescence intensity MFI

Batch

| | NB146 | NB146 | NB147 | NB147 |
|---|---|---|---|---|
| Serum dilution | (TB2) | (TB3) | (TB2) | (TB3) |
| 1:100 | 1857 | 1563 | 1230 | 1811 |
| 1:1000 | 2873 | 2560 | 2393 | 2711 |
| 1:10000 | 592 | 561 | 485 | 628 |

(B) Phage library construction and panning of nanobody against HER2 One week later after four times of immunizations, 100 mL of Vicugna pacos peripheral blood was collected; PBMC was isolated with a lymphocyte separation medium; a total of RNA was extracted with an RNAiso Plus reagent. The extracted RNA was reversely transcribed into cDNA with a PrimeScript™ II 1st Strand cDNA Synthesis Kit (purchased from Takara, Cat. No. 6210A). Nested PCR was applied to amplify the nucleic acid fragment encoding a heavy chain antibody variable region:

The first round of PCRs:

Forward primer:
(SEQ ID NO: 11)
CTTGGTGGTCCTGGCTGC;
Reverse primer:
(SEQ ID NO: 12)
GGTACGTGCTGTTGAACTGTTCC.

The second round of PCRs:

Products from the first round of PCRs were used as templates,

Forward primer:
(SEQ ID NO: 13)
CATGCCATGACTGTGGCCCAGGCGGCCCAGKTGCAGCTCGTGGAGTC;
primer-1:
(SEQ ID NO: 14)
CATGCCATGACTCGCGGCCGGCCTGGCCATGGGGGTCTTCGCTGTGGTG
CG;
Reverse primer-2:
(SEQ ID NO: 15)
CATGCCATGACTCGCGGCCGGCCTGGCCGTCTTGTGGTTTTGGTGTCTT
GGG.

The nucleic acid fragment of the target single-domain antibody was recovered and then cloned into a vector pcomb3XSS used for phage display using a restriction endonuclease SfiI. The product was then electrotransformed into E. coli electroporation competent cells TG1, and a single-domain antibody phage display library against HER2 was constructed and assayed. The size of the reservoir was calculated to be 3.4×10⁹ by gradient dilution plating. To determine the insertion rate of the library, 48 clones were randomly selected and subjected to colony PCR. The results show that the insertion rate is up to 100%.

(C) Panning of the Single-Domain Antibody Against HER2

The human HER2-His protein was diluted by a carbonate buffer solution with a pH value of 9.6 to a final concentration of 5 μg/mL, and added to wells of ELISA plate with 100 μL/well; 8 wells were coated with each protein at 4° C. over the night; the coating buffer was discarded, and the plate was washed with PBS for 3 times; 300 μL, of a 3% BSA-PBS blocking solution was added per well and blocked for 1 h at 37° C.; the plate was washed with PBS for 3 times, and 100 μL of the phage library was added for incubation for 1 h at 37° C.; unbound phage was removed by pipetting; then the plate was washed with PBST for 6 times and washed with PBS twice; 100 μL of Gly-HCl eluent was added to elute specific-binding phage 8 min later after incubation at 37° C.; the eluent was then transferred to 1.5 mL of sterile centrifuge tubes and neutralized with 10 μL, of Tris-HCl neutralizing buffer rapidly; 10 μL, of the neutralized solution was taken and subjected to gradient dilution; titer was assayed and recovery rate of the panning was calculated; the remaining eluates were mixed, then amplified and purified, and could be used for the next round of affinity panning.

192 monoclonals were randomly picked with sterilized toothpicks from an eluate titer plate from the first round of panning, and seeded into 1 mL 2×YT-AK, and subjected to shake cultivation for 8 h at 37° C. and 220 r/min. 100 μL, of the above cultures were taken and M13K07 phages were added by a ratio of cell: phage=1:20, subjected to standing for 15 min at 37° C. and shake cultivation for 45 min at 220 r/min. 300 μL, (volume) of 2×YT-AK was supplemented, and then the mixture was subject to intensive shake cultivation over the night at 30° C. The mixed solution was centrifuged for 2 min at 12000 rpm in the next day; supernatant was taken and used for the identification of monoclonals by ELISA.

The human HER2 protein and monkey HER2 protein were diluted by a carbonate buffer solution with a pH value of 9.6 to final concentrations of 2 μg/mL and 1 μg/mL, and added to wells of ELISA plate with 100 μL/well; the wells were coated at 4° C. over the night; the coating buffer was discarded, and the plate was washed with PBST for 3 times; 300 μL of 5% skimmed milk was added per well for blocking for 1 h at 37° C.; the plate was washed with PBST for 3 times, and 50 μL of the phage culture supernatant and 50 μL of 5% skimmed milk were added for incubation for 1 h at 37° C.; the plate was washed with PBST for 5 times, and horseradish peroxidase-labeled anti-M13 antibodies (diluted by PBS according to 1:10000) were added with 100 μL/well for acting for 1 h at 37° C.; the plate was washed with PBST for 6 times. A TMB color-developing solution was added for color developing with 100 μL/well for 7 min at 37° C., and a stop solution was added to stop the reaction with 50 μL/well, then optical density was measured at a wavelength of 450 nm. Human HER2 positive clones, human HER2-monkey HER2 double positive clones were selected and sent to TSINGKE Biological Technology, and sequenced.

The sequencing results were analyzed and an evolutionary tree was constructed according to the amino acid sequences of the VHH encoded protein; after the sequences close to each other on the evolutionary tree were eliminated according to sequence similarity, the following VHH antibodies (see details in Table 8) were selected to analyze the CDR regions thereof by a bioinformatics method (detailed results are shown in Table 9), and subjected to the subsequent VHH-hFc production and identification. The analysis modes include: Chothia definition and Kabat definition, using the following websites for analysis: hap://cao.labshare.cn/AbRSA/abrsa.php, http://www.abysis.org/abysis/sequence_input/key_annotation/key_annotation.cgi; IMGT definition, using the following web sites for analysis:

http://www.imgt.org/3Dstructure-DB/cgi/DomainGapAlign.cgi#results.

TABLE 8
Sequence information of VHH antibodies

| Antibody name | Sequence information |
|---|---|
| NB146-54 | QLQLVESGGGLVQAGGSLRLSCVASGTFFSTKTMAWYRQAPGNQREWIATISPDG |
| (SEQ ID NO: 16) | TTRHADSMKGRSTISRDNAKKVVYLQLDSLKPEDTAAYYCRDISRDLWGQGTQV |
| | TVSS |
| NB146-125 | EVQLVESGGGLVQAGGSLTLSCVASETIFRRNAMAWYRQAPGQQRELVASIRRGA |
| (SEQ ID NO: 17) | FTYYPNSMKGRFTISRDDAKNTVFLQMNSLKPEDTGVYYCRSLNDDYWGQGTQ |
| | VTVSS |
| NB147-102 | QLQLVESGGGLVQPGGSLRLSCSASGRTFNNYVMAWFRQAAGKEREFVANINTSG |
| (SEQ ID NO: 18) | GRTTYTDSVKDRFTISRDNAKNTMYIQMNNLKAEDTAVYYCAARILYNSDNSDY |
| | RKYYYWGQGTQVTVSS |
| NB147-39 | QLQLVESGGDLVQPGGSLRLSCAGSGFTLDAYAIGWFRQAPGKEREGVSCISSSGG |
| (SEQ ID NO: 19) | TTSYADSVKGRFTISRDYAKNTVYLQMNAVKPEDTAVYYCAIERTCERIGASQFRG |
| | QGTQVTVSV |
| NB147-32 | QLQLVESGGGLVQSGGSLRLSCAASGFSLDYHAIGWFRQAPGKEREGVSCISSSGG |
| (SEQ ID NO: 20) | RTNYADSVKGRFTISRDNAKNTVYLQMNSLKPEDAAVYSCAAVIYNSAWICNLLT |
| | GYEYEYWGQGTQVTVSS |
| NB147-83 | QLQLVESGGGLVQPGGSLTLSCAASGFPASHYYAIRWFRQAPGKERDGIACISSYD |
| (SEQ ID NO: 21) | GSTNYADSVKGRFTISNDGAKKTVYLHMSDVQPEDAAVYFCAATIHFSAYEECQA |
| | YEYHYWGQGTQVTVSS |
| NB147-110 | EVQVVESGGGLVQPGGSLRLSCTASGHTLDYYAIGWFRQAPGKEREGVSCITSTG |
| (SEQ ID NO: 22) | TITNYADSVKGRFTISRDNAKNTVYLQMNSLKPEDTAVYYCAVLRGSYCRSNTED |
| | AWGQGTLVTVSS |
| NB147-66 | QLQLVESGGGLVHTGGSLRLSCAASGSTLDYHAIGWFRQAPGKEREGVSCITSSG |
| (SEQ ID NO: 23) | GRTNYADSVKGRFTVSRDDAKNTVYLQMNSLKPEDTAVYYCAAVIYDSAWICNL |
| | LAGYEYRYWGQGTQVTVSS |
| NB146-171 | EVQLVESGGGLVQPGGSLRLSCAASGFTFSSYPMAWVRQAPGKGEEWVSWISNS |
| (SEQ ID NO: 24) | GGRTTYADSVKGRFTISRDNAKRTVYLQMNKLQPEDTAVYSCTRASGNGERGRG |
| | TQVTVSS |
| NB147-36 | QLQLVESGGGLVQPGGSLRLSCAASGFTLDYHAIGWFRQAPGKEREGVSCISSSG |
| (SEQ ID NO: 25) | GRINYADSVKGRFTISRDMTKNTVYLELNSLKPEDTAIYYCAAEIFDSSWYCPLSR |
| | NNMNYWGKGTLVAVSS |
| NB146-12 | QLQLVESGGGLVQPGGSLRLSCAASGFIFSDTAMNWVRQAPGKGLEWVSSINWS |
| (SEQ ID NO: 26) | GTHTSYADSVKGRFKISRDNAKKALYLQMNSLQPEDTAVYACARGWVDSTRTVV |
| | APLTKGQGTQVTVSS |
| NB147-14 | QVQLVESGGGLVRPGGSLRLSCVLSGRTFSDYAMRWFRQAPGKEREFVASINWSG |
| (SEQ ID NO: 27) | THTDYADSVKGRFTISRDNAKKTVYLQMHSLTPTDTAVYYCTFGWGPQLPGTDY |
| | WGQGTQVTVAP |
| NB146-27 | EVQLVESGGGLAQPGGSLRLSCAASGFRFTSYWMHWVRQAPGKGLEWVSAINT |
| (SEQ ID NO: 28) | GGGSTYYADSVKGRFTISRDNAKNTLYLQMNSLKPEDTALYYCARDLSGSDYVV |
| | GITSWGQGTQVTVSS |
| NB146-62 | EVQVVESGGELVQPGGSLRLSCAASGFSISTYTMTWVRQGPGKGLEWVSTISPLR |
| (SEQ ID NO: 29) | WGQSTTSYADSVKGRFTISRDNAKNTLYLQMNSLNPDDTGVYYCSRPDGKRGQG |
| | TQVTVSS |
| NB147-137 | EVQVVESGGGLVQPGGSLRLSCAASGFTLDYHAIGWFRQAPGKEREGVSCISSSG |
| (SEQ ID NO: 30) | GRTNYADSVKGRFTISRDNAKNTVYLQMNSLKPEDTAVYYCAAEIFDSAWYCPLS |
| | RYDMDYWGKGTLVTVSS |
| NB147-61 | EVQLVESGGGMVQPGGSLRLSCAASGFTLDYYAIGWFRQAPGKEREGVSCISSSG |
| (SEQ ID NO: 31) | GRTNYADSVKGRFTISRDDARNTVYLQMNTLKPEDTAVYYCAAVILDNSWHCGY |
| | SYDMDYWGKGTLVTVSS |
| NB147-161 | QLQLVESGGGWVQAGGSLRLSCAASGFTLDYYAIGWFRQAPGKEREGVSCISSSG |
| (SEQ ID NO: 32) | GRSNYADSVKGRFTISRDNAKNTVYLQMNSLKIEDTGVYYCAAVILDDSWQCGY |
| | YYNMDYWGKGTLVTVSS |
| NB146-28 | EVQLVESGGTRVQPGGSLKLSCATSGIMFSYNTMAWYRQAPGKQRELVATITRDG |
| (SEQ ID NO: 33) | STNYADSMKGRFTISRDNAKNTLYLQMNGLKPEDTAVYYCNLGTTDWRRYNYW |
| | GQGTQVTVSS |

TABLE 9
Information about CDR regions of antibodies

| Antibody | Analysis | | | |
|---|---|---|---|---|
| name | mode | CDR1 | CDR2 | CDR3 |
| NB146-54 | IMGT | GTFFSTKT | ISPDGTT | RDISRDL |
| | | (SEQ ID NO: 34) | (SEQ ID NO: 35) | (SEQ ID NO: 36) |
| | Kabat | TKTMA | TISPDGTTRHADSMKG | ISRDL |
| | | (SEQ ID NO: 37) | (SEQ ID NO: 38) | (SEQ ID NO: 39) |
| | Chothia | GTFFSTK | SPDGT | ISRDL |
| | | (SEQ ID NO: 40) | (SEQ ID NO: 41) | (SEQ ID NO: 42) |
| NB146-125 | IMGT | ETIFRRNA | IRRGAFT | RSLNDDY |
| | | (SEQ ID NO: 43) | (SEQ ID NO: 44) | (SEQ ID NO: 45) |
| | Kabat | RNAMA | SIRRGAFTYYPNSMKG | LNDDY |
| | | (SEQ ID NO: 46) | (SEQ ID NO: 47) | (SEQ ID NO: 48) |
| | Chothia | ETIFRRN | RRGAF | LNDDY |
| | | (SEQ ID NO: 49) | (SEQ ID NO: 50) | (SEQ ID NO: 51) |
| NB147-102 | IMGT | GRTFNNYV | INTSGGRT | AARILYNSDNSDYRKYYY |
| | | (SEQ ID NO: 52) | (SEQ ID NO: 53) | (SEQ ID NO: 54) |
| | Kabat | NYVMA | NINTSGGRTTYTDSVKD | RILYNSDNSDYRKYYY |
| | | (SEQ ID NO: 55) | (SEQ ID NO: 56) | (SEQ ID NO: 57) |
| | Chothia | GRTFNNY | NTSGGR | RILYNSDNSDYRKYYY |
| | | (SEQ ID NO: 58) | (SEQ ID NO: 59) | (SEQ ID NO: 60) |
| NB147-39 | IMGT | GFTLDAYA | ISSSGGTT | AIERTCERIGAS |
| | | (SEQ ID NO: 61) | (SEQ ID NO: 62) | (SEQ ID NO: 63) |
| | Kabat | AYAIG | CISSSGGTTSYADSVKG | ERTCERIGASQF |
| | | (SEQ ID NO: 64) | (SEQ ID NO: 65) | (SEQ ID NO: 66) |
| | Chothia | GFTLDAY | SSSGGT | ERTCERIGASQF |
| | | (SEQ ID NO: 67) | (SEQ ID NO: 68) | (SEQ ID NO: 69) |
| NB147-32 | IMGT | GFSLDYHA | ISSSGGRT | AAVIYNSAWICNLLTGYEYEY |
| | | (SEQ ID NO: 70) | (SEQ ID NO: 71) | (SEQ ID NO: 72) |
| | Kabat | YHAIG | CISSSGGRTNYADSVKG | VIYNSAWICNLLTGYEYEY |
| | | (SEQ ID NO: 73) | (SEQ ID NO: 74) | (SEQ ID NO: 75) |
| | Chothia | GFSLDYH | SSSGGR | VIYNSAWICNLLTGYEYEY |
| | | (SEQ ID NO: 76) | (SEQ ID NO: 77) | (SEQ ID NO: 78) |
| NB147-83 | IMGT | GFPASHYYA | ISSYDGST | AATIHFSAYEECQAYEYHY |
| | | (SEQ ID NO: 79) | (SEQ ID NO: 80) | (SEQ ID NO: 81) |
| | Kabat | HYYAIR | CISSYDGSTNYADSVKG | TIHFSAYEECQAYEYHY |
| | | (SEQ ID NO: 82) | (SEQ ID NO: 83) | (SEQ ID NO: 84) |
| | Chothia | GFPASHYY | SSYDGS | TIHFSAYEECQAYEYHY |
| | | (SEQ ID NO: 85) | (SEQ ID NO: 86) | (SEQ ID NO: 87) |
| NB147-110 | IMGT | GHTLDYYA | ITSTGTIT | AVLRGSYCRSNT |
| | | (SEQ ID NO: 88) | (SEQ ID NO: 89) | (SEQ ID NO: 90) |
| | Kabat | YYAIG | CITSTGTITNYADSVKG | LRGSYCRSNTFDA |
| | | (SEQ ID NO: 91) | (SEQ ID NO: 92) | (SEQ ID NO: 93) |
| | Chothia | GHTLDYY | TSTGTI | LRGSYCRSNTFDA |
| | | (SEQ ID NO: 94) | (SEQ ID NO: 95) | (SEQ ID NO: 96) |
| NB147-66 | IMGT | GSTLDYHA | ITSSGGRT | AAVIYDSAWICNLLAGYEYR |
| | | (SEQ ID NO: 97) | (SEQ ID NO: 98) | Y |
| | | | | (SEQ ID NO: 99) |
| | Kabat | YHAIG | CITSSGGRTNYADSVKG | VIYDSAWICNLLAGYEYRY |
| | | (SEQ ID NO: 100) | (SEQ ID NO: 101) | (SEQ ID NO: 102) |
| | Chothia | GSTLDYH | TSSGGR | VIYDSAWICNLLAGYEYRY |
| | | (SEQ ID NO: 103) | (SEQ ID NO: 104) | (SEQ ID NO: 105) |
| NB146-171 | IMGT | GFTFSSYP | ISNSGGRT | TRASGNGERGRGT |
| | | (SEQ ID NO: 106) | (SEQ ID NO: 107) | (SEQ ID NO: 108) |
| | Kabat | SYPMA | WISNSGGRTTYADSVKG | ASGNGE |
| | | (SEQ ID NO: 109) | (SEQ ID NO: 110) | (SEQ ID NO: 111) |
| | Chothia | GFTFSSY | SNSGGR | ASGNGE |
| | | (SEQ ID NO: 112) | (SEQ ID NO: 113) | (SEQ ID NO: 114) |
| NB147-36 | IMGT | GFTLDYHA | ISSSGGRI | AAEIFDSSWYCPLSRNNMNY |
| | | (SEQ ID NO: 115) | (SEQ ID NO: 116) | (SEQ ID NO: 117) |
| | Kabat | YHAIG | CISSSGGRINYADSVKG | EIFDSSWYCPLSRNNMNY |
| | | (SEQ ID NO: 118) | (SEQ ID NO: 119) | (SEQ ID NO: 120) |
| | Chothia | GFTLDYH | SSSGGR | EIFDSSWYCPLSRNNMNY |
| | | (SEQ ID NO: 121) | (SEQ ID NO: 122) | (SEQ ID NO: 123) |
| NB146-12 | IMGT | GFIFSDTA | INWSGTHT | ARGWVDSTRTVVA |
| | | (SEQ ID NO: 124) | (SEQ ID NO: 125) | (SEQ ID NO: 126) |
| | Kabat | DTAMN | SINWSGTHTSYADSVKG | GWVDSTRTVVAPLT |
| | | (SEQ ID NO: 127) | (SEQ ID NO: 128) | (SEQ ID NO: 129) |
| | Chothia | GFIFSDT | NWSGTH | GWVDSTRTVVAPLT |
| | | (SEQ ID NO: 130) | (SEQ ID NO: 131) | (SEQ ID NO: 132 |
| NB147-14 | IMGT | GRTFSDYA | INWSGTHT | TFGWGPQLPGTDY |
| | | (SEQ ID NO: 133) | (SEQ ID NO: 134) | (SEQ ID NO: 135) |
| | Kabat | DYAMR | SINWSGTHTDYADSVKG | GWGPQLPGTDY |
| | | (SEQ ID NO: 136) | (SEQ ID NO: 137) | (SEQ ID NO: 138) |
| | Chothia | GRTFSDY | NWSGTH | GWGPQLPGTDY |
| | | (SEQ ID NO: 139) | (SEQ ID NO: 140) | (SEQ ID NO: 141) |
| NB146-27 | IMGT | GFRFTSYW | INTGGGST | ARDLSGSDYVVGITS |
| | | (SEQ ID NO: 142) | (SEQ ID NO: 143) | (SEQ ID NO: 144) |
| | Kabat | SYWMH | AINTGGGSTYYADSVKG | DLSGSDYVVGITS |
| | | (SEQ ID NO: 145) | (SEQ ID NO: 146) | (SEQ ID NO: 147) |
| | Chothia | GFRFTSY | NTGGGS | DLSGSDYVVGITS |
| | | (SEQ ID NO: 148) | (SEQ ID NO: 149) | (SEQ ID NO: 150) |
| NB146-62 | IMGT | GFSISTYT | ISPLRWGQST | CSRPDGKRGQGTQ |
| | | (SEQ ID NO: 151) | (SEQ ID NO: 152) | (SEQ ID NO: 153) |
| | Kabat | TYTMT | TISPLRWGQSTTSYADSVK | PDGK |
| | | (SEQ ID NO: 154) | G | (SEQ ID NO: 156) |
| | | | (SEQ ID NO: 155) | |
| | Chothia | GFSISTY | SPLRWGQST | PDGK |
| | | (SEQ ID NO: 157) | (SEQ ID NO: 158) | (SEQ ID NO: 159) |
| NB147-137 | IMGT | GFTLDYHA | ISSSGGRT | AAEIFDSAWYCPLSRYDMDY |
| | | (SEQ ID NO: 160) | (SEQ ID NO: 161) | (SEQ ID NO: 162) |
| | Kabat | YHAIG | CISSSGGRTNYADSVKG | EIFDSAWYCPLSRYDMDY |
| | | (SEQ ID NO: 163) | (SEQ ID NO: 164) | (SEQ ID NO: 165) |
| | Chothia | GFTLDYH | SSSGGR | EIFDSAWYCPLSRYDMDY |
| | | (SEQ ID NO: 166) | (SEQ ID NO: 167) | (SEQ ID NO: 168) |
| NB147-61 | IMGT | GFTLDYYA | ISSSGGRT | AAVILDNSWHCGYSYDMDY |
| | | (SEQ ID NO: 169) | (SEQ ID NO: 170) | (SEQ ID NO: 171) |
| | Kabat | YYAIG | CISSSGGRTNYADSVKG | VILDNSWHCGYSYDMDY |
| | | (SEQ ID NO: 172) | (SEQ ID NO: 173) | (SEQ ID NO: 174) |
| | Chothia | GFTLDYY | SSSGGR | VILDNSWHCGYSYDMDY |
| | | (SEQ ID NO: 175) | (SEQ ID NO: 176) | (SEQ ID NO: 177) |
| NB147-161 | IMGT | GFTLDYYA | ISSSGGRS | AAVILDDSWQCGYYYNMDY |
| | | (SEQ ID NO: 178) | (SEQ ID NO: 179) | (SEQ ID NO: 180) |
| | Kabat | YYAIG | CISSSGGRSNYADSVKG | VILDDSWQCGYYYNMDY |
| | | (SEQ ID NO: 181) | (SEQ ID NO: 182) | (SEQ ID NO: 183) |
| | Chothia | GFTLDYY | SSSGGR | VILDDSWQCGYYYNMDY |
| | | (SEQ ID NO: 184) | (SEQ ID NO: 185) | (SEQ ID NO: 186) |
| NB146-28 | IMGT | GIMFSYNT | ITRDGST | NLGTTDWRRYNY |
| | | (SEQ ID NO: 187) | (SEQ ID NO: 188) | (SEQ ID NO: 189) |
| | Kabat | YNTMA | TITRDGSTNYADSMKG | GTTDWRRYNY |
| | | (SEQ ID NO: 190) | (SEQ ID NO: 191) | (SEQ ID NO: 192 |
| | Chothia | GIMFSYN | TRDGS | GTTDWRRYNY |
| | | (SEQ ID NO: 193) | (SEQ ID NO: 194) | (SEQ ID NO: 195) |

### Example 3. Production of VHH-hFc

Target VHH sequences were recombined into expression vectors of human IgG1 Fc to obtain recombinant plasmids. Detailed plasmid construction, transfection and purification processes are referring to Example 1 (A).

The purified VHH-hFc was subjected to assays and analysis on protein concentration, purity and endotoxin (a Lonza kit). Results are shown in Table 10. The results show that the antibodies had a high purity of final products and the endotoxin concentration was within 1.0 EU/mg.

TABLE 10
Quality control of the purified VHH-hFc antibodies

| | | Antibody | Purity of | |
|---|---|---|---|---|
| | | concen- | antibody | |
| | | tration | (SEC, | Endotoxin |
| Antibody name | Clone No. | (mg/mL) | 280 nm) % | EU/mg |
| NB146-171 | NB146-171 | 0.85 | 99.23 | <1 |
| NB146-27 | NB146-27 | 0.31 | 99.47 | <1 |
| NB146-62 | NB146-62 | 0.75 | 93.18 | <1 |
| NB146-12 | NB146-12 | 0.42 | 92.06 | <1 |
| NB146-54 | NB146-54 | 0.88 | 99.01 | <1 |
| NB146-28 | NB146-28 | 0.73 | 99.69 | <1 |
| NB146-125 | NB146-125 | 0.96 | 99.60 | <1 |
| NB147-83 | NB147-83 | 0.97 | 92.80 | <1 |
| NB147-102 | NB147-102 | 0.69 | 98.92 | <1 |
| NB147-110 | NB147-110 | 0.73 | 98.54 | <1 |
| NB147-137 | NB147-137 | 0.94 | 98.49 | <1 |
| NB147-14 | NB147-14 | 0.76 | 90.48 | <1 |
| NB147-161 | NB147-161 | 0.87 | 98.88 | <1 |
| NB147-32 | NB147-32 | 1.58 | 99.29 | <1 |
| NB147-36 | NB147-36 | 2.96 | 89.42 | <1 |
| NB147-39 | NB147-39 | 2.46 | 98.87 | <1 |
| NB147-61 | NB147-61 | 2.52 | 98.71 | <1 |
| NB147-66 | NB147-66 | 1.62 | 97.52 | <1 |

### Example 4. Identification of VHH-hFc

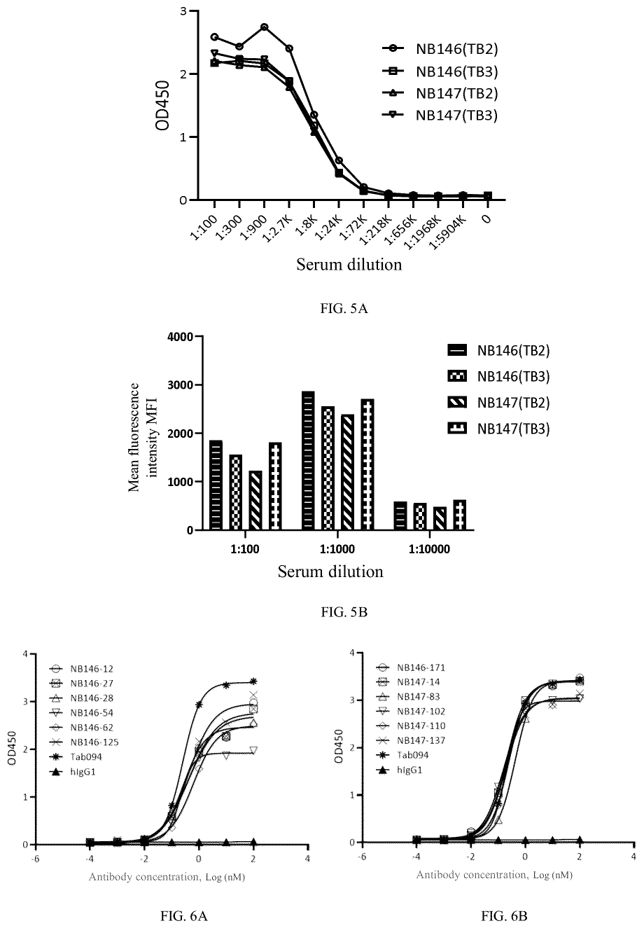

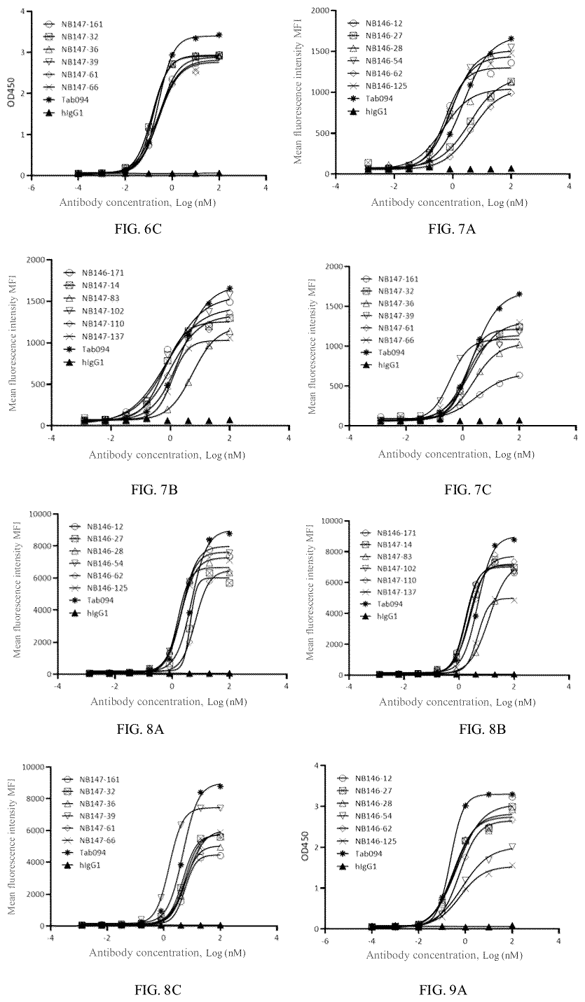

(A) Assay on binding of VHH-hFc to a human HER2 protein by enzyme-linked immunosorbent assay (ELISA) A human HER2 protein was diluted with PBS to a final concentration of 2 μg/mL, and then added to a 96-well ELISA plate with 50 μL/well. The plate was sealed with a plastic film and incubated at 4° C. over the night, washed twice with PBS in the next day, added with a blocking solution [PBS+2% (w/w) BSA] and blocked at room temperature for 2 h. The blocking solution was discarded, and 100 nM VHH-hFc (diluted in a gradient), or a control antibody were added with 50 μL/well. After incubation for 2 h at 37° C., the plate was washed for 3 times with PBS. A secondary antibody labeled with HRP (horseradish peroxidase) (purchased from Sigma, Cat. No. A0170) was added. After incubation for 1 h at 37° C., the plate was washed for 5 times with PBS. A TMB substrate was added with 50 μL/well and incubated at room temperature for 10 min, then a stop solution (1.0 M HCl) was added with 50 μL/well. The OD_{450 nm} values were read by an ELISA plate reader (Multimode Plate Reader, EnSight, purchased from Perkin Elmer). Results of the binding activity of VHH-Fc with human HER2 protein are shown in FIGS. 6A-6C and Table 11, indicating that all the purified antibodies bind to human HER2 protein. IgG control is hIgG1, and data in the table are values of OD_{450 nm}; N/A denotes no data here.

TABLE 11
Assay results for binding reactions of VHH-hFc antibodies with a human HER2 protein by
ELISA

| | Antibody concentration (nM) | | | | | | | |
|---|---|---|---|---|---|---|---|---|
| Antibody name | 100 | 10 | 1 | 0.1 | 0.01 | 0.001 | 0.0001 | 0 |
| NB146-12 | 2.98 | 2.25 | 2.02 | 0.54 | 0.09 | 0.05 | 0.05 | 0.05 |
| NB146-27 | 2.85 | 2.27 | 1.95 | 0.59 | 0.11 | 0.05 | 0.05 | 0.05 |
| NB146-28 | 2.56 | 2.30 | 2.03 | 0.65 | 0.10 | 0.05 | 0.05 | 0.05 |
| NB146-54 | 1.97 | 1.87 | 1.82 | 0.45 | 0.09 | 0.05 | 0.05 | 0.05 |
| NB146-62 | 2.53 | 2.30 | 1.59 | 0.36 | 0.08 | 0.05 | 0.05 | 0.05 |
| NB146-125 | 3.14 | 2.56 | 2.17 | 0.58 | 0.11 | 0.11 | 0.06 | 0.06 |
| NB146-171 | 3.47 | 3.30 | 2.93 | 0.99 | 0.24 | 0.07 | 0.06 | 0.05 |
| NB147-14 | 3.40 | 3.34 | 3.00 | 1.05 | 0.14 | 0.06 | 0.06 | 0.05 |
| NB147-83 | 3.43 | 3.33 | 2.61 | 0.48 | 0.10 | 0.06 | 0.07 | 0.05 |
| NB147-102 | 3.03 | 3.00 | 2.73 | 1.17 | 0.20 | 0.06 | 0.05 | 0.05 |
| NB147-110 | 3.05 | 2.90 | 2.83 | 0.79 | 0.14 | 0.06 | 0.05 | 0.05 |
| NB147-137 | 3.14 | 2.91 | 2.70 | 1.06 | 0.17 | 0.08 | 0.05 | 0.05 |
| NB147-161 | 2.95 | 2.73 | 2.34 | 0.74 | 0.12 | 0.06 | 0.05 | 0.05 |
| NB147-32 | 2.93 | 2.88 | 2.72 | 1.19 | 0.20 | 0.07 | 0.05 | 0.05 |
| NB147-36 | 2.91 | 2.92 | 2.71 | 1.18 | 0.19 | 0.07 | 0.05 | 0.05 |
| NB147-39 | 2.88 | 2.91 | 2.71 | 1.06 | 0.17 | 0.06 | 0.05 | 0.05 |
| NB147-61 | 2.89 | 2.53 | 2.23 | 0.76 | 0.12 | 0.06 | 0.05 | 0.05 |
| NB147-66 | 2.94 | 2.56 | 2.28 | 0.85 | 0.15 | 0.06 | 0.05 | 0.05 |
| Tab094 | 3.43 | 3.34 | 2.94 | 0.82 | 0.12 | 0.06 | 0.05 | 0.08 |
| hIgG1 | 0.06 | 0.05 | 0.05 | 0.05 | 0.06 | 0.05 | 0.06 | N/A |

(B) Assay on Binding of the Antibodies with Cells Expressing HER2 Protein by Flow Cytometry Assay (FACS)

The desired cells were expanded to a logarithmic growth phase in a T-75 cell culture flask. The culture medium was removed by pipetting, and the cells were washed twice with a PBS buffer, digested with trypsin, and the digestion was stopped by a complete medium; and the cells were blown up to obtain a single-cell suspension. The cells were centrifuged after cell counting, and the cell pellet was resuspended to 2×10⁶ cells/mL with FACS buffer (PBS+2% (w/w) FBS) and added to a 96-well FACS reaction plate with 50 μL/well. The VHH-hFc or control antibody was added with 50 μL/well, and the plate was incubated at 4° C. for 1 h. After the plate was centrifuged and washed for 3 times with a PBS buffer, an FITC-labeled secondary antibody (purchased from Invitrogen, Cat. No. A18830) was added with 50 μL/well, and the plate was incubated on ice for 1 h. After the plate was centrifuged and washed for 3 times with a PBS buffer, the results of 100 μl were assayed and analyzed by FACS (FACS Canto™, purchased from BD Biosciences). Data analysis was performed by software (FlowJo) to obtain the mean fluorescence intensity (MFI) of the cells. Analysis was then performed by software (GraphPad Prism8), data were fitted, and EC₅₀ values were calculated. The analysis results are shown in Table 12, FIGS. 7A-7C (VHH-hFc and CHO-K1-human HER2) and FIGS. 8A-8C (VHH-hFc and SK-BR-3), indicating that all the VHH-hFc could bind to CHO-K1-human HER2 cells and SK-BR-3 cells.

TABLE 12
Assay results for binding reactions of VHH-
hFc with cells expressing human HER2 by FACS

| | SK-BR-3 | | CHO-K1-human HER2 | |
|---|---|---|---|---|
| | Maximum | | Maximum | |
| | mean | | mean | |
| | fluorescence | | fluorescence | |
| Antibody | intensity | | intensity | |
| name | Max MFI | Ec50(nM) | Max MFI | Ec50(nM) |
| NB146-12 | 7770 | 2.11 | 1358 | 0.56 |
| NB146-27 | 6312 | 4.12 | 1131 | 3.81 |
| NB146-28 | 6945 | 1.54 | 1119 | 0.46 |
| NB146-54 | 8260 | 2.17 | 1546 | 0.90 |
| NB146-62 | 6386 | 6.36 | 990 | 4.47 |
| NB146-125 | 7290 | 1.97 | 1476 | 0.80 |
| NB146-171 | 7350 | 1.66 | 1490 | 0.65 |
| NB147-14 | 7289 | 2.25 | 1298 | 0.59 |
| NB147-83 | 6785 | 12.06 | 1140 | 5.57 |
| NB147-102 | 7490 | 1.81 | 1577 | 0.92 |
| NB147-110 | 7792 | 2.70 | 1363 | 1.04 |
| NB147-137 | 4922 | 4.75 | 1061 | 1.13 |
| NB147-161 | 4412 | 5.50 | 638 | 3.11 |
| NB147-32 | 5593 | 4.78 | 1242 | 1.36 |
| NB147-36 | 4948 | 5.35 | 1022 | 2.36 |
| NB147-39 | 7400 | 1.56 | 1185 | 0.36 |
| NB147-61 | 5677 | 5.64 | 1187 | 1.29 |
| NB147-66 | 5889 | 6.71 | 1301 | 1.97 |
| Tab094 | 8775 | 4.77 | 1656 | 4.83 |
| hIgG1 | 73 | No | 71 | No |
| | | binding | | binding |

### Example 5. Assay on Cross-Binding Activity of VHH-hFc

(A) Assay on Binding of VHH-hFc with Monkey HER2 Protein and Murine HER2 Protein by ELISA

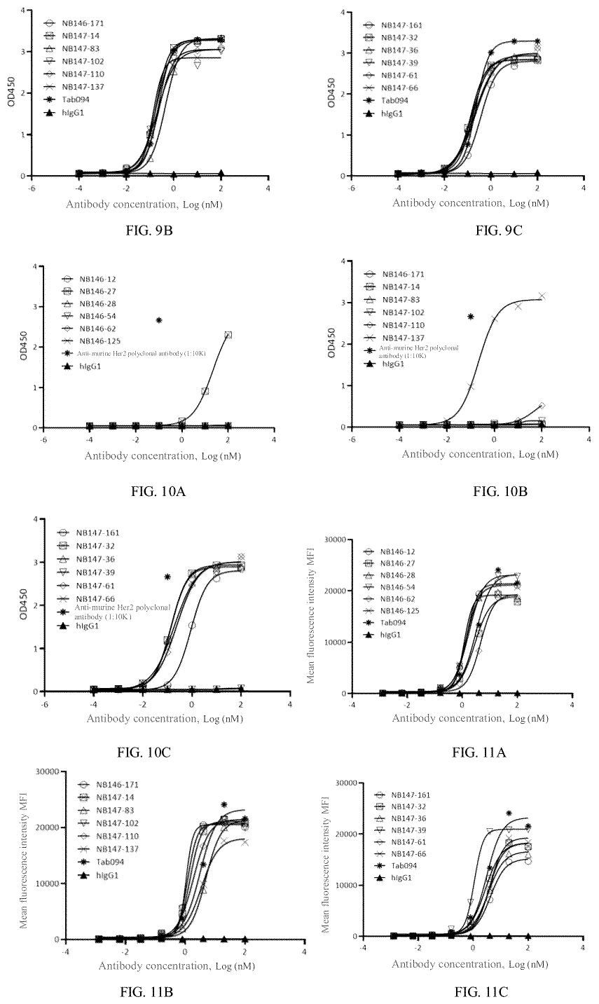

The monkey HER2-His protein (purchased from Sino Biological, Cat. No. 90295-C08H) and murine HER2-His protein (purchased from Sino Biological, Cat. No. 50714-M08H) were assayed by ELISA and subjected to data analysis according to the method as described in Example 4 (A). The analysis results are shown in FIGS. 9A-9C and 10A-10C, and Tables 13-14, indicating that all the VHH-hFc could bind to monkey HER2 protein, and 7 VHH-hFc could bind to murine HER2 protein. IgG control is hIgG1, and data in the table are values of OD_{450 nm}; N/A denotes no data here.

TABLE 13
Assay results for binding reactions of VHH-hFc antibodies with monkey HER2 protein by
ELISA

| | Antibody concentration (nM) | | | | | | | |
|---|---|---|---|---|---|---|---|---|
| Antibody name | 100 | 10 | 1 | 0.1 | 0.01 | 0.001 | 0.0001 | Blank |
| NB146-12 | 3.23 | 2.49 | 2.16 | 0.60 | 0.12 | 0.06 | 0.05 | 0.05 |
| NB146-27 | 2.99 | 2.46 | 2.16 | 0.65 | 0.11 | 0.06 | 0.05 | 0.05 |
| NB146-28 | 2.93 | 2.41 | 2.18 | 0.71 | 0.12 | 0.06 | 0.05 | 0.05 |
| NB 146-54 | 2.01 | 1.69 | 1.19 | 0.36 | 0.08 | 0.05 | 0.05 | 0.05 |
| NB146-62 | 2.67 | 2.50 | 1.76 | 0.40 | 0.09 | 0.06 | 0.05 | 0.05 |
| NB146-125 | 1.56 | 1.35 | 0.99 | 0.30 | 0.08 | 0.08 | 0.06 | 0.06 |
| NB146-171 | 3.32 | 3.20 | 3.00 | 1.04 | 0.20 | 0.08 | 0.07 | 0.05 |
| NB147-14 | 3.31 | 3.25 | 3.10 | 1.00 | 0.15 | 0.07 | 0.05 | 0.06 |
| NB147-83 | 3.32 | 3.27 | 2.52 | 0.43 | 0.10 | 0.06 | 0.06 | 0.05 |
| NB147-102 | 3.01 | 2.66 | 2.83 | 1.13 | 0.18 | 0.07 | 0.06 | 0.06 |
| NB147-110 | 3.06 | 3.02 | 2.72 | 0.79 | 0.12 | 0.06 | 0.06 | 0.06 |
| NB147-137 | 3.18 | 2.85 | 2.65 | 0.98 | 0.16 | 0.06 | 0.05 | 0.06 |
| NB147-161 | 2.87 | 2.68 | 2.23 | 0.51 | 0.08 | 0.05 | 0.05 | 0.05 |
| NB147-32 | 2.83 | 2.83 | 2.70 | 1.19 | 0.19 | 0.06 | 0.05 | 0.05 |
| NB147-36 | 2.93 | 2.91 | 2.66 | 1.16 | 0.19 | 0.06 | 0.05 | 0.05 |
| NB147-39 | 2.82 | 2.84 | 2.66 | 1.01 | 0.16 | 0.06 | 0.05 | 0.05 |
| NB147-61 | 3.12 | 2.77 | 2.49 | 0.97 | 0.16 | 0.06 | 0.05 | 0.05 |
| NB147-66 | 3.13 | 2.74 | 2.53 | 1.06 | 0.16 | 0.07 | 0.05 | 0.06 |
| Tab094 | 3.29 | 3.29 | 3.02 | 0.77 | 0.12 | 0.07 | 0.07 | 0.07 |
| hIgG1 | 0.09 | 0.05 | 0.05 | 0.06 | 0.05 | 0.09 | 0.06 | N/A |

TABLE 14
Assay results for binding reactions of VHH-hFc antibodies with murine HER2 protein by ELISA

| | Antibody concentration (nM) | | | | | | | |
|---|---|---|---|---|---|---|---|---|
| Antibody name | 100 | 10 | 1 | 0.1 | 0.01 | 0.001 | 0.0001 | 0 |
| NB146-12 | 0.05 | 0.05 | 0.05 | 0.05 | 0.05 | 0.05 | 0.05 | 0.05 |
| NB146-27 | 2.31 | 0.91 | 0.17 | 0.06 | 0.05 | 0.05 | 0.05 | 0.05 |
| NB146-28 | 0.05 | 0.05 | 0.05 | 0.05 | 0.05 | 0.05 | 0.05 | 0.05 |
| NB146-54 | 0.05 | 0.05 | 0.05 | 0.05 | 0.05 | 0.05 | 0.05 | 0.05 |
| NB146-62 | 0.05 | 0.05 | 0.05 | 0.05 | 0.05 | 0.05 | 0.05 | 0.05 |
| NB146-125 | 0.06 | 0.06 | 0.06 | 0.05 | 0.05 | 0.05 | 0.05 | 0.06 |
| NB146-171 | 0.06 | 0.05 | 0.05 | 0.05 | 0.05 | 0.05 | 0.05 | 0.06 |
| NB147-14 | 0.07 | 0.05 | 0.09 | 0.05 | 0.05 | 0.05 | 0.05 | 0.05 |
| NB147-83 | 0.07 | 0.06 | 0.05 | 0.05 | 0.05 | 0.06 | 0.05 | 0.05 |
| NB147-102 | 0.15 | 0.07 | 0.05 | 0.06 | 0.05 | 0.06 | 0.05 | 0.05 |
| NB147-110 | 0.52 | 0.16 | 0.06 | 0.05 | 0.05 | 0.06 | 0.05 | 0.05 |
| NB147-137 | 3.17 | 2.91 | 2.62 | 0.98 | 0.16 | 0.07 | 0.05 | 0.05 |
| NB147-161 | 2.85 | 2.63 | 1.54 | 0.15 | 0.05 | 0.05 | 0.05 | 0.05 |
| NB147-32 | 2.92 | 2.93 | 2.75 | 1.20 | 0.20 | 0.06 | 0.05 | 0.05 |
| NB147-36 | 2.89 | 2.87 | 2.72 | 1.17 | 0.19 | 0.07 | 0.05 | 0.05 |
| NB147-39 | 0.08 | 0.05 | 0.05 | 0.05 | 0.05 | 0.05 | 0.05 | 0.05 |
| NB147-61 | 3.13 | 2.78 | 2.47 | 0.91 | 0.14 | 0.06 | 0.05 | 0.05 |
| NB147-66 | 3.13 | 2.80 | 2.52 | 1.08 | 0.18 | 0.06 | 0.05 | 0.05 |
| hIgG1 | 0.07 | 0.05 | 0.05 | 0.05 | 0.05 | 0.05 | 0.05 | N/A |

| | | |
|---|---|---|
| Anti-murine Her2 | 2.41 | *The antibody is a polyclonal antibody; the reaction concentration |
| polyclonal antibody | | is a concentration after being diluted by 1:10,000 |
| (1:10K) | | |

(B) Assay on Binding of VHH-hFc with Cells Expressing Monkey HER2 Protein by ELISA

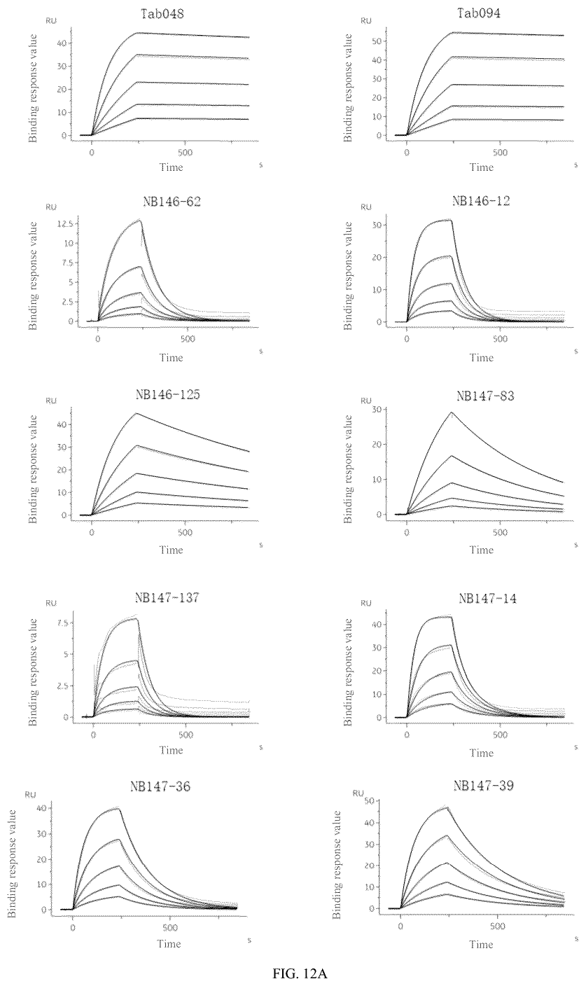

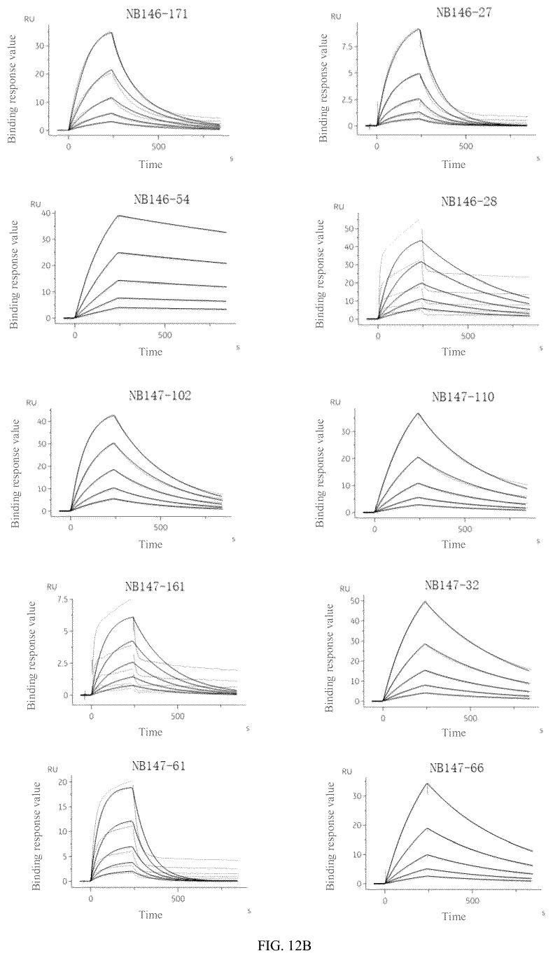

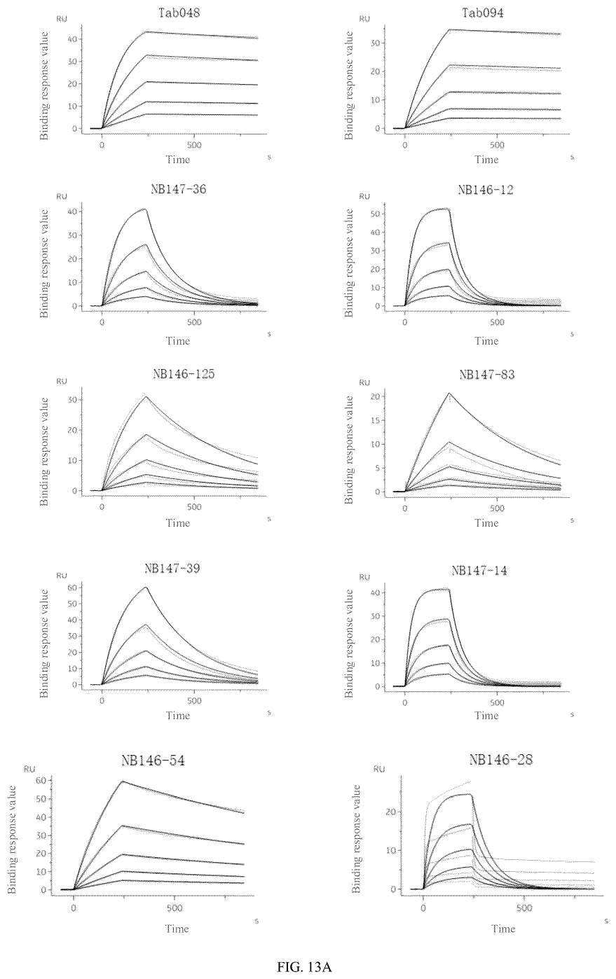

HEK293T-monkey HER2 cells were subjected to FACS assay and data analysis according to the method described in Example 4 (B). The analysis results are shown in Table 15 and FIGS. 11A-11C, indicating that all the VHH-hFc have binding activity to the HEK293T-monkey-HER2 cell.

TABLE 15
Assay results for binding reactions of VHH-hFc
with cells expressing monkey HER2 by FACS

HEK293T-monkey-HER2

| | Maximum mean | |
|---|---|---|
| | fluorescence intensity | |
| Antibody name | Max MFI | Ec50(nM) |
| NB146-12 | 21622 | 1.40 |
| NB146-27 | 19327 | 2.81 |
| NB146-28 | 19760 | 1.15 |
| NB146-54 | 22959 | 1.71 |
| NB146-62 | 18789 | 4.62 |
| NB146-125 | 21297 | 1.40 |
| NB146-171 | 20847 | 1.05 |
| NB147-14 | 21318 | 1.43 |
| NB147-83 | 20827 | 4.82 |
| NB147-102 | 21430 | 1.20 |
| NB147-110 | 21517 | 1.87 |
| NB147-137 | 17644 | 3.39 |
| NB147-161 | 14670 | 4.19 |
| NB147-32 | 18292 | 2.80 |
| NB147-36 | 16140 | 3.48 |
| NB147-39 | 20898 | 1.09 |
| NB147-61 | 18360 | 3.79 |
| NB147-66 | 19221 | 4.02 |
| Tab094 | 24096 | 4.61 |
| hIgG1 | 127 | No binding |

### Example 6. Assay on Affinity of HER2-hFc

(A) Assay on Affinity of VHH-hFc with Human HER2 Protein

The anti-human HER2 VHH-hFc antibodies were captured using a Protein A chip (GE Healthcare; 29-127-558). The sample and running buffer were HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then a single concentration of HER2 antigen protein was injected. The association and dissociation processes of the antibody with the antigen protein were recorded, and finally, the chip was regenerated using Glycine pH 1.5 (GE Healthcare; BR-1003-54). The association was determined by injecting different concentrations of recombinant human HER2-His in the solution and maintaining for 240 s, wherein the flow rate was 30 μL/min, and the protein was diluted in a 1:1 dilution ratio from 200 nM (see detailed results for actual concentrations tested) to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections were also subtracted (=double referencing). To calculate the apparent KD and other kinetic parameters, the Langmuir 1:1 model was used. The association rate (Kₐ), dissociation rate (K_{dis}), and binding affinity (1(13) of the VHH-hFc with the HER2 protein are shown in the table, in which the antibody Tab094 is used as a control. As shown in Table 16 and FIGS. 12A-12B, all the VHH-hFc have good affinity with human HER2 protein, of which the minimum affinity was around 1E-07 M and the optimal affinity was around 6E-09 M.

TABLE 16
Binding affinity of VHH-hFc antibodies with human HER2 protein

| Antibody name | Kₐ (1/Ms) | K_{dis} (1/s) | KD (M) |
|---|---|---|---|
| NB146-125 | 6.82E+04 | 7.89E−04 | 1.16E−08 |
| NB147-36 | 1.14E+05 | 6.39E−03 | 5.61E−08 |
| NB147-39 | 1.12E+05 | 3.57E−03 | 3.18E−08 |
| NB147-83 | 3.01E+04 | 2.08E−03 | 6.90E−08 |
| NB146-171 | 3.81E+07 | 2.91E+00 | 7.64E−08 |
| NB147-61 | 9.53E+04 | 1.08E−02 | 1.13E−07 |
| NB147-102 | 2.23E+05 | 3.61E−03 | 1.62E−08 |
| NB146-27 | 2.08E+04 | 8.78E−03 | 4.23E−07 |
| NB147-66 | 2.19E+04 | 2.04E−03 | 9.30E−08 |
| NB147-110 | 5.59E+04 | 4.38E−03 | 7.85E−08 |
| NB146-62 | 2.62E+04 | 1.09E−02 | 4.18E−07 |
| NB147-137 | 5.41E+04 | 1.44E−02 | 2.67E−07 |
| NB146-12 | 2.61E+05 | 1.45E−02 | 5.54E−08 |
| NB147-14 | 6.71E+05 | 1.99E−02 | 2.97E−08 |
| NB146-54 | 4.83E+04 | 3.01E−04 | 6.22E−09 |
| NB147-161 | 9.37E+04 | 4.65E−03 | 4.96E−08 |
| NB146-28 | 1.94E+05 | 2.20E−03 | 1.13E−08 |
| NB147-32 | 2.91E+04 | 2.00E−03 | 6.89E−08 |
| Tab048 | 2.21E+05 | 7.45E−05 | 3.37E−10 |
| Tab094 | 9.96E+04 | 4.57E−05 | 4.59E−10 |

(B) Assay on Affinity of HER2 VHH-hFc Antibodies with Monkey HER2-his Protein

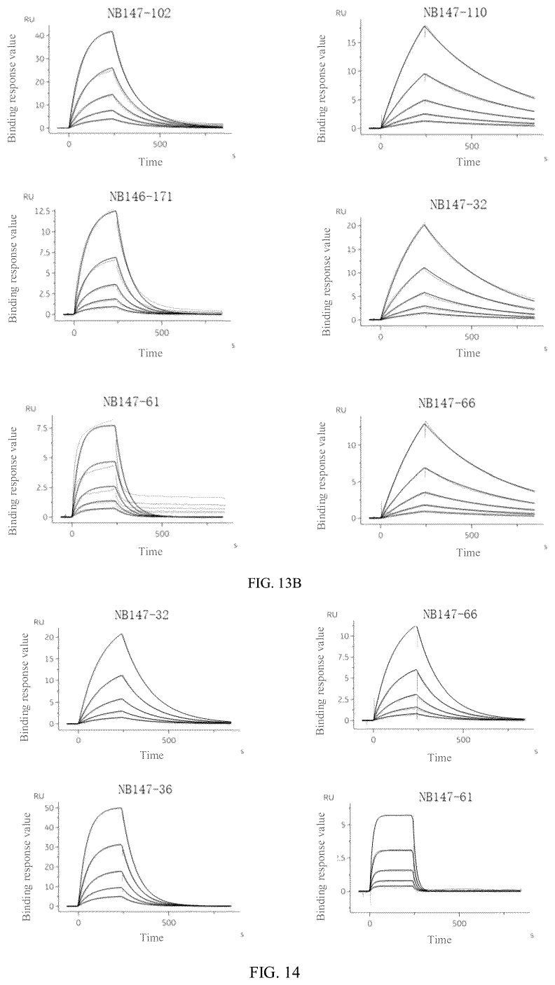

Affinity of VHH-hFc antibodies with monkey HER2-his protein was assayed according to the method described in Example 6 (A), of which antibodies Tab048 and Tab094 served as controls. As shown in Table 17 and FIGS. 13A-13B, all the VHH-hFc antibodies have better affinity with monkey HER2-his protein, other than NB146-27, NB146-62, NB147-137 and NB147-161.

TABLE 17
Binding affinity of VHH-hFc antibodies
with monkey HER2 protein

| Antibody name | ka (1/Ms) | kd (1/s) | KD (M) |
|---|---|---|---|
| NB146-125 | 7.41E+04 | 2.11E−03 | 2.84E−08 |
| NB147-36 | 3.27E+05 | 1.45E−02 | 4.42E−08 |
| NB147-39 | 9.81E+04 | 3.79E−03 | 3.86E−08 |
| NB146-171 | 6.36E+04 | 1.21E−02 | 1.90E−07 |
| NB147-102 | 3.46E+05 | 1.92E−02 | 5.54E−08 |
| NB147-66 | 7.40E+04 | 4.39E−03 | 5.93E−08 |
| NB147-110 | 7.69E+04 | 4.17E−03 | 5.42E−08 |
| NB146-12 | 5.80E+05 | 3.10E−02 | 5.35E−08 |
| NB147-14 | 4.34E+05 | 1.64E−02 | 3.78E−08 |
| NB146-54 | 6.65E+04 | 5.68E−04 | 8.53E−09 |
| NB146-28 | 2.95E+05 | 1.15E−02 | 3.88E−08 |
| NB147-32 | 4.09E+04 | 2.79E−03 | 6.83E−08 |
| NB147-83 | 1.27E+03 | 2.18E−03 | 1.71E−06 |
| NB147-61 | 1.90E+05 | 1.73E−02 | 9.11E−08 |

| | |
|---|---|
| NB146-27 | No binding |
| NB146-62 | No binding |
| NB147-137 | No binding |
| NB147-161 | No binding |

| | | | |
|---|---|---|---|
| Tab048 | 1.91E+05 | 1.18E−04 | 6.17E-10 |
| Tab094 | 9.69E+04 | 8.23E−05 | 8.49E-10 |

(C) Assay on Affinity of HER2 VHH-hFc with Murine HER2-his Protein

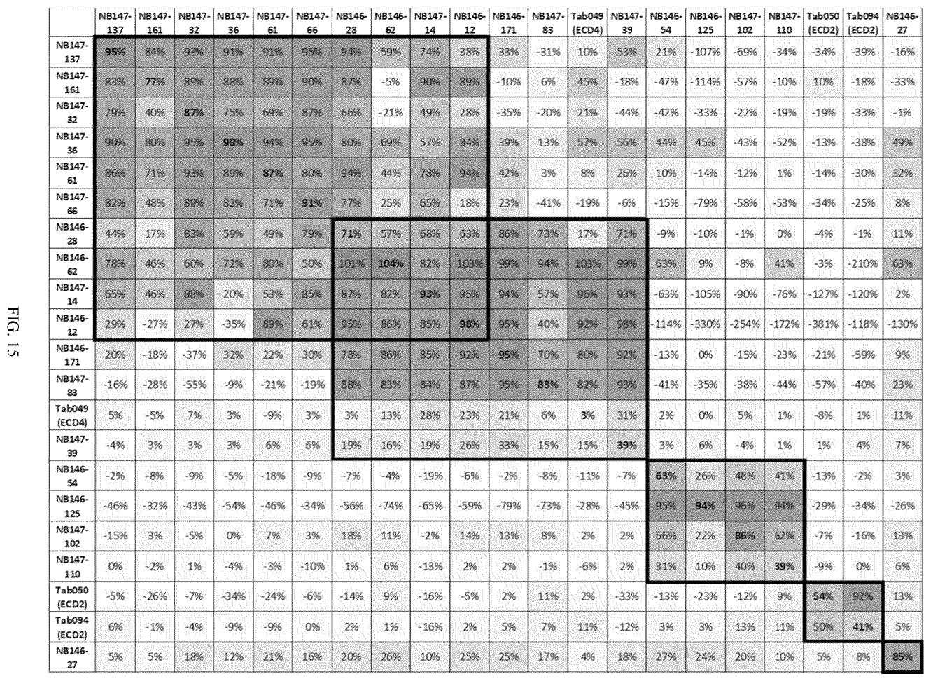

Affinity of VHH-hFc antibodies with murine HER2-his protein was assayed according to the method described in Example 6 (A), of which the antibody Tab094 served as a control. As shown in Table 18 and FIG. 14, NB147-32, NB147-36, NB147-66 and NB147-61 have better affinity with murine HER2-his protein.

TABLE 18
Binding affinity of VHH-hFc antibodies with murine HER2 protein

| Antibody name | ka (1/Ms) | kd (1/s) | KD (M) |
|---|---|---|---|
| NB147-32 | 1.81E+04 | 6.77E−03 | 3.74E−07 |
| NB147-36 | 9.26E+04 | 1.25E−02 | 1.35E−07 |
| NB147-66 | 1.59E+04 | 7.44E−03 | 4.67E−07 |
| NB147-61 | 8.30E+04 | 5.69E−02 | 6.85E−07 |

| | |
|---|---|
| NB146-27 | No binding |
| NB147-137 | No binding |
| NB147-161 | No binding |

### Example 7. Antibody-Antigen Binding Epitope Binning

To identify the antigen-binding sites to antibodies, HER2 VHH-hFc antibodies were grouped by competitive ELISA. ELISA plate was coated with 2 μg/mL VHH-hFc according to the method described in Example 4 (A); the human HER2 protein was subjected to gradient dilution from 30 μg/mL, and EC₈₀ was calculated as the concentration in competitive ELISA.

VHH-hFc was diluted by PBS to 2 μg/mL and coated on a 96-well high adsorption ELISA plate with 50 μL/well, after the plate was coated at 4° C. overnight and then blocked with 250 μL blocking solution (PBS containing 2% (w/w) BSA) for 2 h at room temperature, after 40 μg/mL of the antibodies to be tested were added, human HER2-his protein with an EC₈₀ concentration corresponding to each of the antibodies to be tested was then added for incubation for 2 h, after the plate was washed with PBS for 5 times, HRP-labeled anti-His secondary antibody was added and incubated for 1 h, and then the plate was washed for 5 times. A TMB substrate was added with 50 μL/well and incubated at room temperature for 10 min, then a stop solution (1.0 M HCl) was added with 50 μL/well. OD_{450 nm} values were read by an ELISA plate reader (Insight, purchased from Perkin Elmer). Based on the OD_{450 nm} values, a competitive rate between antibodies was calculated by a formula (inhibition ratio=(OD_{450 nm} value of negative control−OD_{450 nm} value of competitive antibody sample)*100%/OD_{450 nm} value of negative control). Results are shown in FIG. 15; the higher the value of the competitive rate is, the closer the antigen epitope bound to the two antibodies is.

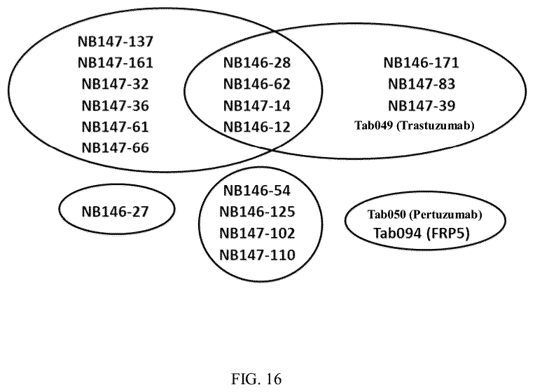

Based on the competitive rate, the VHH antibodies were classified. The results are shown in FIG. 16. NB146-28, NB146-62, NB147-14, NB146-12, NB147-171, NB147-83 and NB147-39 were competitive with Tab049 (Trastuzumab) and thus, classified as a same category; NB146-28, NB146-62, NB147-14, NB146-12, NB147-137, NB147-161, NB147-32, NB147-36, NB147-61 and NB147-66 were competitive with each other and thus, classified as a same category; NB146-54, NB146-125, NB147-102 and NB147-110 were competitive with each other and thus, classified as a same category; NB146-27 was not competitive with other antibodies and thus, was classified as a separate category; Tab050 (Pertuzumab) and Tab094 (FRP5 monoclonal antibody) were competitive with each other and thus, classified as a same category.

1. An antibody or an antigen-binding fragment specifically binding to Her2, wherein the antibody or the antigen-binding fragment comprises a CDR1, a CDR2 and a CDR3; the CDR1, the CDR2 and the CDR3 comprise an HCDR1, an HCDR2 and an HCDR3 in a VHH domain set forth in any one of SEQ ID NOs: 16-33, respectively.

2. The antibody or the antigen-binding fragment according to claim 1, wherein the HCDR1, the HCDR2 and the HCDR3 are determined according to an IMGT numbering scheme, a Kabat numbering scheme or a Chothia numbering scheme; optionally, the HCDR1, the HCDR2 and the HCDR3 are selected from Table 9;
optionally, the HCDR1 is selected from SEQ ID NOs: 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190 and 193;
optionally, the HCDR2 is selected from SEQ ID NOs: 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191 and 194;
optionally, the HCDR3 is selected from SEQ ID NOs: 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192 and 195;
preferably, the HCDR1, the HCDR2 and the HCDR3 are selected from SEQ ID NOs: 34-36, SEQ ID NOs: 37-39 and SEQ ID NOs: 40-42 according to the IMGT numbering scheme, the Kabat numbering scheme or the Chothia numbering scheme;
preferably, the HCDR1, the HCDR2 and the HCDR3 are selected from SEQ ID NOs: 43-45, SEQ ID NOs: 46-48 and SEQ ID NOs: 49-51 according to the IMGT numbering scheme, the Kabat numbering scheme or the Chothia numbering scheme;
preferably, the HCDR1, the HCDR2 and the HCDR3 are selected from SEQ ID NOs: 52-54, SEQ ID NOs: 55-57 and SEQ ID NOs: 58-60 according to the IMGT numbering scheme, the Kabat numbering scheme or the Chothia numbering scheme;
preferably, the HCDR1, the HCDR2 and the HCDR3 are selected from SEQ ID NOs: 61-63, SEQ ID NOs: 64-66 and SEQ ID NOs: 67-69 according to the IMGT numbering scheme, the Kabat numbering scheme or the Chothia numbering scheme;
preferably, the HCDR1, the HCDR2 and the HCDR3 are selected from SEQ ID NOs: 70-72, SEQ ID NOs: 73-75 and SEQ ID NOs: 76-78 according to the IMGT numbering scheme, the Kabat numbering scheme or the Chothia numbering scheme;
preferably, the HCDR1, the HCDR2 and the HCDR3 are selected from SEQ ID NOs: 79-81, SEQ ID NOs: 82-84 and SEQ ID NOs: 85-87 according to the IMGT numbering scheme, the Kabat numbering scheme or the Chothia numbering scheme;
preferably, the HCDR1, the HCDR2 and the HCDR3 are selected from SEQ ID NOs: 88-90, SEQ ID NOs: 91-93 and SEQ ID NOs: 94-96 according to the IMGT numbering scheme, the Kabat numbering scheme or the Chothia numbering scheme;
preferably, the HCDR1, the HCDR2 and the HCDR3 are selected from SEQ ID NOs: 97-99, SEQ ID NOs: 100-102 and SEQ ID NOs: 103-105 according to the IMGT numbering scheme, the Kabat numbering scheme or the Chothia numbering scheme;
preferably, the HCDR1, the HCDR2 and the HCDR3 are selected from SEQ ID NOs: 106-108, SEQ ID NOs: 109-111 and SEQ ID NOs: 112-114 according to the IMGT numbering scheme, the Kabat numbering scheme or the Chothia numbering scheme;
preferably, the HCDR1, the HCDR2 and the HCDR3 are selected from SEQ ID NOs: 115-117, SEQ ID NOs: 118-120 and SEQ ID NOs: 121-123 according to the IMGT numbering scheme, the Kabat numbering scheme or the Chothia numbering scheme;
preferably, the HCDR1, the HCDR2 and the HCDR3 are selected from SEQ ID NOs: 124-126, SEQ ID NOs: 127-129 and SEQ ID NOs: 130-132 according to the IMGT numbering scheme, the Kabat numbering scheme or the Chothia numbering scheme;
preferably, the HCDR1, the HCDR2 and the HCDR3 are selected from SEQ ID NOs: 133-135, SEQ ID NOs: 136-138 and SEQ ID NOs: 139-141 according to the IMGT numbering scheme, the Kabat numbering scheme or the Chothia numbering scheme;
preferably, the HCDR1, the HCDR2 and the HCDR3 are selected from SEQ ID NOs: 142-144, SEQ ID NOs: 145-147 and SEQ ID NOs: 148-150 according to the IMGT numbering scheme, the Kabat numbering scheme or the Chothia numbering scheme;
preferably, the HCDR1, the HCDR2 and the HCDR3 are selected from SEQ ID NOs: 151-153, SEQ ID NOs: 154-156 and SEQ ID NOs: 157-159 according to the IMGT numbering scheme, the Kabat numbering scheme or the Chothia numbering scheme;
preferably, the HCDR1, the HCDR2 and the HCDR3 are selected from SEQ ID NOs: 160-162, SEQ ID NOs: 163-165 and SEQ ID NOs: 166-168 according to the IMGT numbering scheme, the Kabat numbering scheme or the Chothia numbering scheme;
preferably, the HCDR1, the HCDR2 and the HCDR3 are selected from SEQ ID NOs: 169-171, SEQ ID NOs: 172-174 and SEQ ID NOs: 175-177 according to the IMGT numbering scheme, the Kabat numbering scheme or the Chothia numbering scheme;
preferably, the HCDR1, the HCDR2 and the HCDR3 are selected from SEQ ID NOs: 178-180, SEQ ID NOs: 181-183 and SEQ ID NOs: 184-186 according to the IMGT numbering scheme, the Kabat numbering scheme or the Chothia numbering scheme;
preferably, the HCDR1, the HCDR2 and the HCDR3 are selected from SEQ ID NOs: 187-189, SEQ ID NOs: 190-192 and SEQ ID NOs: 193-195 according to the IMGT numbering scheme, the Kabat numbering scheme or the Chothia numbering scheme.

3. The antibody or the antigen-binding fragment according to claim 1, wherein the CDR1, the CDR2, and/or the CDR3 comprise amino acid sequences having at most 10, 9, 8, 7, 6, 5, 4, 3, 2, or 1 mutation on the HCDR1, the HCDR2, and/or the HCDR3; the mutation is selected from an insertion, a deletion, and/or a substitution; the substitution is preferably a substitution of conserved amino acids.

4. The antibody or the antigen-binding fragment according to claim 1, wherein the CDR1, the CDR2, and/or the CDR3 comprise sequences that is at least 80%, 85%, 90%, 91%, 92%, 93%, 94%, 95%, 96%, 97%, 98%, 99%, or 100% identical to the HCDR1, the HCDR2, and/or the HCDR3, respectively.

5. The antibody or the antigen-binding fragment according to claim 1, wherein the antibody or the antigen-binding fragment comprises a single-domain antibody comprising the CDR1, the CDR2, and the CDR3;
optionally, wherein the single-domain antibody comprises a sequence set forth in any one of SEQ ID NOs: 16-33; optionally, the single-domain antibody comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 16-33; the mutation is selected from an insertion, a deletion, and/or a substitution; the substitution is preferably a substitution of conserved amino acids; optionally, the single-domain antibody comprises a sequence that is at least 80%, 85%, 90%, 91%, 92%, 93%, 94%, 95%, 96%, 97%, 98%, 99%, or 100% identical to the sequence set forth in any one of SEQ ID NOs: 16-33.

6. (canceled)

7. The antibody or the antigen-binding fragment according to claim 1, wherein the antibody comprises an FR region in the VHH domain set forth in any one of SEQ ID NOs: 16-33;
optionally, the antibody comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 16-33; the mutation is selected from an insertion, a deletion, and/or a substitution; the substitution is preferably a substitution of conserved amino acids;
optionally, the antibody comprises a sequence that is at least 80%, 85%, 90%, 91%, 92%, 93%, 94%, 95%, 96%, 97%, 98%, 99%, or 100% identical to the FR region in the VHH domain set forth in any one of SEQ ID NOs: 16-33.

8. The antibody or the antigen-binding fragment according to claim 1, wherein the antibody or the antigen-binding fragment is: (1) a chimeric antibody or a fragment thereof, (2) a humanized antibody or a fragment thereof, or (3) a fully human antibody or a fragment thereof;
optionally, wherein the antibody or the antigen-binding fragment comprises or does not comprise an antibody heavy chain constant region; optionally, the antibody heavy chain constant region is selected from human, Vicugna pacos, mouse, rat, rabbit and sheep; optionally, the antibody heavy chain constant region is selected from IgG, IgM, IgA, IgE and IgD, and the IgG is selected from IgG1, IgG2, IgG3 and IgG4; optionally, the antibody heavy chain constant region is selected from an Fc region, a CH3 region and an intact heavy chain constant region; preferably, the heavy chain constant region is a human Fc region; and preferably, the antibody or the antigen-binding fragment is a heavy chain antibody;
optionally, wherein the antibody or the antigen-binding fragment is further conjugated to a therapeutic agent or a tracer, wherein preferably, the therapeutic agent is selected from a radioisotope, a chemotherapeutic agent, and an immunomodulator, and the tracer is selected from a radiocontrast medium, a paramagnetic ion, a metal, a fluorescent label, a chemiluminescent label, an ultrasound contrast agent, and a photosensitizer
optionally, wherein the antibody or the antigen-binding fragment specifically binds to human HER2, monkey HER2 and/or murine HER2; preferably, the antibody or the antigen-binding fragment binds to human HER2, monkey HER2 and/or murine HER2 with a KD value of less than 1E-6 M, 1E-7 M, 2E-7 M, 3E-7 M, 4E-7 M, 5E-7 M, 6E-7 M, 8E-7 M, 9E-7 M, 1E-8 M, 2E-8 M, 3E-8 M, 4E-8 M, 5E-8 M, 6E-8 M, 8E-8 M, 9E-8 M; 1E-9 M, 2E-9 M, 3E-9 M, 4E-9 M, 5E-9 M, 6E-9 M, 8E-9 M, 9E-9 M, 1E-10 M or 1E-11 M
optionally, wherein the antibody or the antigen-binding fragment competitively binds to or does not competitively bind to HER2 with Trastuzumab, Pertuzumab or an FRP5 monoclonal antibody
optionally, wherein the antibody or the antigen-binding fragment is further linked with an additional functional molecule; preferably, the additional functional molecule is selected from one or more of a signal peptide, a protein tag, a cytokine, an angiogenesis inhibitor and an immune checkpoint inhibitor;
optionally, wherein the cytokine is IL2, IL-6, IL-12, IL-15, IL-21, IFN or TNF-alpha; the angiogenesis inhibitor is endostatin; and the immune checkpoint inhibitor is SIRPα.

9-14. (canceled)

15. A multispecific antigen-binding molecule, wherein the multispecific antigen-binding molecule comprises the antibody or the antigen-binding fragment according to claim 1, and an antigen-binding molecule binding to antigens other than HER2 or binding to an HER2 epitope different from the antibody or the antigen-binding fragment; optionally, the other antigens other than HER2 are selected from: CD3, preferably CD3 c; CD16, preferably CD16A; CD137; CD258; 4-1BB; CD40; CD64; EGFR; HER1; HER3; PD-1; PD-L1; VEGF; IGF-IR (insulin-like growth factor-I receptor); phosphatidylserine (PS); c-Met; and a blood-brain barrier receptor;
preferably, the additional antigen-binding molecule is an antibody or antigen-binding fragment;
preferably, the multispecific antigen-binding molecule is a bispecific antigen-binding molecule, a trispecific antigen-binding molecule, or a tetraspecific antigen-binding molecule; and
preferably, the multispecific antigen-binding molecule is divalent, tetravalent or hexavalent.

16. A chimeric antigen receptor, wherein the chimeric antigen receptor comprises an extracellular antigen-binding domain, a transmembrane domain, and an intracellular signaling domain; the extracellular antigen-binding domain comprises the antibody or the antigen-binding fragment according to claim 1.

17. An immune effector cell, wherein the immune effector cell expresses the chimeric antigen receptor according to claim 16 or comprises a nucleic acid fragment encoding the chimeric antigen receptor according to claim 16; preferably, the immune effector cell is selected from a T cell, a natural killer (NK) cell, a natural killer T (NKT) cell, a double negative T (DNT) cell, a monocyte, a macrophage, a dendritic cell and a mastocyte; the T cell is preferably selected from a cytotoxic T cell, a regulatory T cell and a helper T cell; preferably, the immune effector cell is an autologous immune effector cell or an allogeneic immune effector cell.

18. An isolated nucleic acid fragment, wherein the isolated nucleic acid fragment encodes the antibody or the antigen-binding fragment according to claim 1.

19. A vector, wherein the vector comprises the nucleic acid fragment according to claim 18.

20. A host cell, wherein the host cell comprises the vector according to claim 19, and preferably, the cell is a prokaryotic cell or a eukaryotic cell, e.g., a bacterium (E. coli), a fungus (yeast), an insect cell, or a mammalian cell (CHO cell line or 293T cell line).

21. A method for preparing the antibody or the antigen-binding fragment according to claim 1, wherein the method comprises culturing a, and isolating an antibody, an antigen-binding fragment or a multispecific antigen-binding molecule expressed by the cell, wherein the cell is a host cell comprising an isolated nucleic acid fragment, and the isolated nucleic acid fragment encodes the antibody or the antigen-binding fragment.

22. A method for preparing an immune effector cell, wherein the method comprises introducing a nucleic acid fragment encoding the chimeric antigen receptor according to claim 16 into the immune effector cell, and optionally, the method further comprises initiating expression of the chimeric antigen receptor according to claim 16 by the immune effector cell.

23. A pharmaceutical composition, wherein the pharmaceutical composition comprises the antibody or the antigen-binding fragment according to claim 1; optionally, the pharmaceutical composition further comprises a pharmaceutically acceptable carrier, a diluent, or an adjuvant; and optionally, the pharmaceutical composition further comprises an additional antineoplastic agent.

24. A method for the treatment of a tumor or a cancer, wherein the method comprises administering to a subject an effective amount of the antibody or the antigen-binding fragment according to claim 1; preferably, the tumor or the cancer is selected from a solid tumor, gastric carcinoma, gastroesophageal junction carcinoma, ovarian cancer, fallopian tube cancer, peritoneal cancer, endometrial cancer, prostatic cancer, castration resistant prostate cancer, breast cancer, HER2-positive breast cancer, sarcoma, osteosarcoma, glioblastoma multiforme, lung cancer, non-small cell lung cancer, cholangiocarcinoma, urothelium carcinoma, bladder cancer, esophageal cancer, colorectal cancer, head and neck cancers, salivary gland cancer and B-cell acute lymphocytic leukemia.

25-26. (canceled)

27. A kit, wherein the kit comprises the antibody or the antigen-binding fragment according to claim 1.

28. A method for determining expression of HER2 in a biological sample, wherein the method comprises contacting the biological sample with the antibody or the antigen-binding fragment according to claim 1 under such conditions that the antibody or the antigen-binding fragment according to and HER2 form a complex; preferably, the method further comprises determining the formation of the complex and indicating the existence or an expression level of HER2 in the sample.

29. A method for preparing an HER2 assay regent, wherein the method comprises using the antibody or the antigen-binding fragment according to claim 1.


SEQUENCE LISTING (195 sequences)

SEQ ID NO: 1 - protein, 449 aa, artificial sequence
  Heavy chain of Tab048
QVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, artificial sequence
  Light chain of Tab048
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, artificial sequence
  Heavy chain of Tab049
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, artificial sequence
  Light chain of Tab049
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 449 aa, artificial sequence
  Heavy chain of Tab050
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, artificial sequence
  Light chain of Tab050
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 473 aa, artificial sequence
  single chain of Tab094
QVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 232 aa, artificial sequence
  hFc
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 330 aa, artificial sequence
  CH(humam IgG)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, artificial sequence [sequence omitted]

SEQ ID NO: 11 - DNA, 18 bp, artificial sequence
  forward primer
cttggtggtcctggctgc

SEQ ID NO: 12 - DNA, 23 bp, artificial sequence
  reverse primer
ggtacgtgctgttgaactgttcc

SEQ ID NO: 13 - DNA, 47 bp, artificial sequence
  forward primer
catgccatgactgtggcccaggcggcccagktgcagctcgtggagtc

SEQ ID NO: 14 - DNA, 51 bp, artificial sequence
  reverse primer
catgccatgactcgcggccggcctggccatgggggtcttcgctgtggtgcg

SEQ ID NO: 15 - DNA, 52 bp, artificial sequence
  reverse primer
catgccatgactcgcggccggcctggccgtcttgtggttttggtgtcttggg

SEQ ID NO: 16 - protein, 113 aa, Vicugna pacos
QLQLVESGGGLVQAGGSLRLSCVASGTFFSTKTMAWYRQAPGNQREWIATISPDGTTRHADSMKGRSTISRDNAKKVVYLQLDSLKPEDTAAYYCRDISRDLWGQGTQVTVSS

SEQ ID NO: 17 - protein, 113 aa, Vicugna pacos
EVQLVESGGGLVQAGGSLTLSCVASETIFRRNAMAWYRQAPGQQRELVASIRRGAFTYYPNSMKGRFTISRDDAKNTVFLQMNSLKPEDTGVYYCRSLNDDYWGQGTQVTVSS

SEQ ID NO: 18 - protein, 125 aa, Vicugna pacos
QLQLVESGGGLVQPGGSLRLSCSASGRTFNNYVMAWFRQAAGKEREFVANINTSGGRTTYTDSVKDRFTISRDNAKNTMYIQMNNLKAEDTAVYYCAARILYNSDNSDYRKYYYWGQGTQVTVSS

SEQ ID NO: 19 - protein, 121 aa, Vicugna pacos
QLQLVESGGDLVQPGGSLRLSCAGSGFTLDAYAIGWFRQAPGKEREGVSCISSSGGTTSYADSVKGRFTISRDYAKNTVYLQMNAVKPEDTAVYYCAIERTCERIGASQFRGQGTQVTVSV

SEQ ID NO: 20 - protein, 128 aa, Vicugna pacos
QLQLVESGGGLVQSGGSLRLSCAASGFSLDYHAIGWFRQAPGKEREGVSCISSSGGRTNYADSVKGRFTISRDNAKNTVYLQMNSLKPEDAAVYSCAAVIYNSAWICNLLTGYEYEYWGQGTQVTVSS

SEQ ID NO: 21 - protein, 127 aa, Vicugna pacos
QLQLVESGGGLVQPGGSLTLSCAASGFPASHYYAIRWFRQAPGKERDGIACISSYDGSTNYADSVKGRFTISNDGAKKTVYLHMSDVQPEDAAVYFCAATIHFSAYEECQAYEYHYWGQGTQVTVSS

SEQ ID NO: 22 - protein, 122 aa, Vicugna pacos
EVQVVESGGGLVQPGGSLRLSCTASGHTLDYYAIGWFRQAPGKEREGVSCITSTGTITNYADSVKGRFTISRDNAKNTVYLQMNSLKPEDTAVYYCAVLRGSYCRSNTFDAWGQGTLVTVSS

SEQ ID NO: 23 - protein, 128 aa, Vicugna pacos
QLQLVESGGGLVHTGGSLRLSCAASGSTLDYHAIGWFRQAPGKEREGVSCITSSGGRTNYADSVKGRFTVSRDDAKNTVYLQMNSLKPEDTAVYYCAAVIYDSAWICNLLAGYEYRYWGQGTQVTVSS

SEQ ID NO: 24 - protein, 115 aa, Vicugna pacos
EVQLVESGGGLVQPGGSLRLSCAASGFTFSSYPMAWVRQAPGKGEEWVSWISNSGGRTTYADSVKGRFTISRDNAKRTVYLQMNKLQPEDTAVYSCTRASGNGERGRGTQVTVSS

SEQ ID NO: 25 - protein, 127 aa, Vicugna pacos
QLQLVESGGGLVQPGGSLRLSCAASGFTLDYHAIGWFRQAPGKEREGVSCISSSGGRINYADSVKGRFTISRDMTKNTVYLELNSLKPEDTAIYYCAAEIFDSSWYCPLSRNNMNYWGKGTLVAVSS

SEQ ID NO: 26 - protein, 123 aa, Vicugna pacos
QLQLVESGGGLVQPGGSLRLSCAASGFIFSDTAMNWVRQAPGKGLEWVSSINWSGTHTSYADSVKGRFKISRDNAKKALYLQMNSLQPEDTAVYACARGWVDSTRTVVAPLTKGQGTQVTVSS

SEQ ID NO: 27 - protein, 120 aa, Vicugna pacos
QVQLVESGGGLVRPGGSLRLSCVLSGRTFSDYAMRWFRQAPGKEREFVASINWSGTHTDYADSVKGRFTISRDNAKKTVYLQMHSLTPTDTAVYYCTFGWGPQLPGTDYWGQGTQVTVAP

SEQ ID NO: 28 - protein, 122 aa, Vicugna pacos
EVQLVESGGGLAQPGGSLRLSCAASGFRFTSYWMHWVRQAPGKGLEWVSAINTGGGSTYYADSVKGRFTISRDNAKNTLYLQMNSLKPEDTALYYCARDLSGSDYVVGITSWGQGTQVTVSS

SEQ ID NO: 29 - protein, 116 aa, Vicugna pacos
EVQVVESGGELVQPGGSLRLSCAASGFSISTYTMTWVRQGPGKGLEWVSTISPLRWGQSTTSYADSVKGRFTISRDNAKNTLYLQMNSLNPDDTGVYYCSRPDGKRGQGTQVTVSS

SEQ ID NO: 30 - protein, 127 aa, Vicugna pacos
EVQVVESGGGLVQPGGSLRLSCAASGFTLDYHAIGWFRQAPGKEREGVSCISSSGGRTNYADSVKGRFTISRDNAKNTVYLQMNSLKPEDTAVYYCAAEIFDSAWYCPLSRYDMDYWGKGTLVTVSS

SEQ ID NO: 31 - protein, 126 aa, Vicugna pacos
EVQLVESGGGMVQPGGSLRLSCAASGFTLDYYAIGWFRQAPGKEREGVSCISSSGGRTNYADSVKGRFTISRDDARNTVYLQMNTLKPEDTAVYYCAAVILDNSWHCGYSYDMDYWGKGTLVTVSS

SEQ ID NO: 32 - protein, 126 aa, Vicugna pacos
QLQLVESGGGWVQAGGSLRLSCAASGFTLDYYAIGWFRQAPGKEREGVSCISSSGGRSNYADSVKGRFTISRDNAKNTVYLQMNSLKIEDTGVYYCAAVILDDSWQCGYYYNMDYWGKGTLVTVSS

SEQ ID NO: 33 - protein, 118 aa, Vicugna pacos
EVQLVESGGTRVQPGGSLKLSCATSGIMFSYNTMAWYRQAPGKQRELVATITRDGSTNYADSMKGRFTISRDNAKNTLYLQMNGLKPEDTAVYYCNLGTTDWRRYNYWGQGTQVTVSS

SEQ ID NO: 34 - protein, 8 aa, artificial sequence
  CDR
GTFFSTKT

SEQ ID NO: 35 - protein, 7 aa, artificial sequence
  CDR
ISPDGTT

SEQ ID NO: 36 - protein, 7 aa, artificial sequence
  CDR
RDISRDL

SEQ ID NO: 37 - protein, 5 aa, artificial sequence
  CDR
TKTMA

SEQ ID NO: 38 - protein, 16 aa, artificial sequence
  CDR
TISPDGTTRHADSMKG

SEQ ID NO: 39 - protein, 5 aa, artificial sequence
  CDR
ISRDL

SEQ ID NO: 40 - protein, 7 aa, artificial sequence
  CDR
GTFFSTK

SEQ ID NO: 41 - protein, 5 aa, artificial sequence
  CDR
SPDGT

SEQ ID NO: 42 - protein, 5 aa, artificial sequence
  CDR
ISRDL

SEQ ID NO: 43 - protein, 8 aa, artificial sequence
  CDR
ETIFRRNA

SEQ ID NO: 44 - protein, 7 aa, artificial sequence
  CDR
IRRGAFT

SEQ ID NO: 45 - protein, 7 aa, artificial sequence
  CDR
RSLNDDY

SEQ ID NO: 46 - protein, 5 aa, artificial sequence
  CDR
RNAMA

SEQ ID NO: 47 - protein, 16 aa, artificial sequence
  CDR
SIRRGAFTYYPNSMKG

SEQ ID NO: 48 - protein, 5 aa, artificial sequence
  CDR
LNDDY

SEQ ID NO: 49 - protein, 7 aa, artificial sequence
  CDR
ETIFRRN

SEQ ID NO: 50 - protein, 5 aa, artificial sequence
  CDR
RRGAF

SEQ ID NO: 51 - protein, 5 aa, artificial sequence
  CDR
LNDDY

SEQ ID NO: 52 - protein, 8 aa, artificial sequence
  CDR
GRTFNNYV

SEQ ID NO: 53 - protein, 8 aa, artificial sequence
  CDR
INTSGGRT

SEQ ID NO: 54 - protein, 18 aa, artificial sequence
  CDR
AARILYNSDNSDYRKYYY

SEQ ID NO: 55 - protein, 5 aa, artificial sequence
  CDR
NYVMA

SEQ ID NO: 56 - protein, 17 aa, artificial sequence
  CDR
NINTSGGRTTYTDSVKD

SEQ ID NO: 57 - protein, 16 aa, artificial sequence
  CDR
RILYNSDNSDYRKYYY

SEQ ID NO: 58 - protein, 7 aa, artificial sequence
  CDR
GRTFNNY

SEQ ID NO: 59 - protein, 6 aa, artificial sequence
  CDR
NTSGGR

SEQ ID NO: 60 - protein, 16 aa, artificial sequence
  CDR
RILYNSDNSDYRKYYY

SEQ ID NO: 61 - protein, 8 aa, artificial sequence
  CDR
GFTLDAYA

SEQ ID NO: 62 - protein, 8 aa, artificial sequence
  CDR
ISSSGGTT

SEQ ID NO: 63 - protein, 12 aa, artificial sequence
  CDR
AIERTCERIGAS

SEQ ID NO: 64 - protein, 5 aa, artificial sequence
  CDR
AYAIG

SEQ ID NO: 65 - protein, 17 aa, artificial sequence
  CDR
CISSSGGTTSYADSVKG

SEQ ID NO: 66 - protein, 12 aa, artificial sequence
  CDR
ERTCERIGASQF

SEQ ID NO: 67 - protein, 7 aa, artificial sequence
  CDR
GFTLDAY

SEQ ID NO: 68 - protein, 6 aa, artificial sequence
  CDR
SSSGGT

SEQ ID NO: 69 - protein, 12 aa, artificial sequence
  CDR
ERTCERIGASQF

SEQ ID NO: 70 - protein, 8 aa, artificial sequence
  CDR
GFSLDYHA

SEQ ID NO: 71 - protein, 8 aa, artificial sequence
  CDR
ISSSGGRT

SEQ ID NO: 72 - protein, 21 aa, artificial sequence
  CDR
AAVIYNSAWICNLLTGYEYEY

SEQ ID NO: 73 - protein, 5 aa, artificial sequence
  CDR
YHAIG

SEQ ID NO: 74 - protein, 17 aa, artificial sequence
  CDR
CISSSGGRTNYADSVKG

SEQ ID NO: 75 - protein, 19 aa, artificial sequence
  CDR
VIYNSAWICNLLTGYEYEY

SEQ ID NO: 76 - protein, 7 aa, artificial sequence
  CDR
GFSLDYH

SEQ ID NO: 77 - protein, 6 aa, artificial sequence
  CDR
SSSGGR

SEQ ID NO: 78 - protein, 19 aa, artificial sequence
  CDR
VIYNSAWICNLLTGYEYEY

SEQ ID NO: 79 - protein, 9 aa, artificial sequence
  CDR
GFPASHYYA

SEQ ID NO: 80 - protein, 8 aa, artificial sequence
  CDR
ISSYDGST

SEQ ID NO: 81 - protein, 19 aa, artificial sequence
  CDR
AATIHFSAYEECQAYEYHY

SEQ ID NO: 82 - protein, 6 aa, artificial sequence
  CDR
HYYAIR

SEQ ID NO: 83 - protein, 17 aa, artificial sequence
  CDR
CISSYDGSTNYADSVKG

SEQ ID NO: 84 - protein, 17 aa, artificial sequence
  CDR
TIHFSAYEECQAYEYHY

SEQ ID NO: 85 - protein, 8 aa, artificial sequence
  CDR
GFPASHYY

SEQ ID NO: 86 - protein, 6 aa, artificial sequence
  CDR
SSYDGS

SEQ ID NO: 87 - protein, 17 aa, artificial sequence
  CDR
TIHFSAYEECQAYEYHY

SEQ ID NO: 88 - protein, 8 aa, artificial sequence
  CDR
GHTLDYYA

SEQ ID NO: 89 - protein, 8 aa, artificial sequence
  CDR
ITSTGTIT

SEQ ID NO: 90 - protein, 12 aa, artificial sequence
  CDR
AVLRGSYCRSNT

SEQ ID NO: 91 - protein, 5 aa, artificial sequence
  CDR
YYAIG

SEQ ID NO: 92 - protein, 17 aa, artificial sequence
  CDR
CITSTGTITNYADSVKG

SEQ ID NO: 93 - protein, 13 aa, artificial sequence
  CDR
LRGSYCRSNTFDA

SEQ ID NO: 94 - protein, 7 aa, artificial sequence
  CDR
GHTLDYY

SEQ ID NO: 95 - protein, 6 aa, artificial sequence
  CDR
TSTGTI

SEQ ID NO: 96 - protein, 13 aa, artificial sequence
  CDR
LRGSYCRSNTFDA

SEQ ID NO: 97 - protein, 8 aa, artificial sequence
  CDR
GSTLDYHA

SEQ ID NO: 98 - protein, 8 aa, artificial sequence
  CDR
ITSSGGRT

SEQ ID NO: 99 - protein, 21 aa, artificial sequence
  CDR
AAVIYDSAWICNLLAGYEYRY

SEQ ID NO: 100 - protein, 5 aa, artificial sequence
  CDR
YHAIG

SEQ ID NO: 101 - protein, 17 aa, artificial sequence
  CDR
CITSSGGRTNYADSVKG

SEQ ID NO: 102 - protein, 19 aa, artificial sequence
  CDR
VIYDSAWICNLLAGYEYRY

SEQ ID NO: 103 - protein, 7 aa, artificial sequence
  CDR
GSTLDYH

SEQ ID NO: 104 - protein, 6 aa, artificial sequence
  CDR
TSSGGR

SEQ ID NO: 105 - protein, 19 aa, artificial sequence
  CDR
VIYDSAWICNLLAGYEYRY

SEQ ID NO: 106 - protein, 8 aa, artificial sequence
  CDR
GFTFSSYP

SEQ ID NO: 107 - protein, 8 aa, artificial sequence
  CDR
ISNSGGRT

SEQ ID NO: 108 - protein, 13 aa, artificial sequence
  CDR
TRASGNGERGRGT

SEQ ID NO: 109 - protein, 5 aa, artificial sequence
  CDR
SYPMA

SEQ ID NO: 110 - protein, 17 aa, artificial sequence
  CDR
WISNSGGRTTYADSVKG

SEQ ID NO: 111 - protein, 6 aa, artificial sequence
  CDR
ASGNGE

SEQ ID NO: 112 - protein, 7 aa, artificial sequence
  CDR
GFTFSSY

SEQ ID NO: 113 - protein, 6 aa, artificial sequence
  CDR
SNSGGR

SEQ ID NO: 114 - protein, 6 aa, artificial sequence
  CDR
ASGNGE

SEQ ID NO: 115 - protein, 8 aa, artificial sequence
  CDR
GFTLDYHA

SEQ ID NO: 116 - protein, 8 aa, artificial sequence
  CDR
ISSSGGRI

SEQ ID NO: 117 - protein, 20 aa, artificial sequence
  CDR
AAEIFDSSWYCPLSRNNMNY

SEQ ID NO: 118 - protein, 5 aa, artificial sequence
  CDR
YHAIG

SEQ ID NO: 119 - protein, 17 aa, artificial sequence
  CDR
CISSSGGRINYADSVKG

SEQ ID NO: 120 - protein, 18 aa, artificial sequence
  CDR
EIFDSSWYCPLSRNNMNY

SEQ ID NO: 121 - protein, 7 aa, artificial sequence
  CDR
GFTLDYH

SEQ ID NO: 122 - protein, 6 aa, artificial sequence
  CDR
SSSGGR

SEQ ID NO: 123 - protein, 18 aa, artificial sequence
  CDR
EIFDSSWYCPLSRNNMNY

SEQ ID NO: 124 - protein, 8 aa, artificial sequence
  CDR
GFIFSDTA

SEQ ID NO: 125 - protein, 8 aa, artificial sequence
  CDR
INWSGTHT

SEQ ID NO: 126 - protein, 13 aa, artificial sequence
  CDR
ARGWVDSTRTVVA

SEQ ID NO: 127 - protein, 5 aa, artificial sequence
  CDR
DTAMN

SEQ ID NO: 128 - protein, 17 aa, artificial sequence
  CDR
SINWSGTHTSYADSVKG

SEQ ID NO: 129 - protein, 14 aa, artificial sequence
  CDR
GWVDSTRTVVAPLT

SEQ ID NO: 130 - protein, 7 aa, artificial sequence
  CDR
GFIFSDT

SEQ ID NO: 131 - protein, 6 aa, artificial sequence
  CDR
NWSGTH

SEQ ID NO: 132 - protein, 14 aa, artificial sequence
  CDR
GWVDSTRTVVAPLT

SEQ ID NO: 133 - protein, 8 aa, artificial sequence
  CDR
GRTFSDYA

SEQ ID NO: 134 - protein, 8 aa, artificial sequence
  CDR
INWSGTHT

SEQ ID NO: 135 - protein, 13 aa, artificial sequence
  CDR
TFGWGPQLPGTDY

SEQ ID NO: 136 - protein, 5 aa, artificial sequence
  CDR
DYAMR

SEQ ID NO: 137 - protein, 17 aa, artificial sequence
  CDR
SINWSGTHTDYADSVKG

SEQ ID NO: 138 - protein, 11 aa, artificial sequence
  CDR
GWGPQLPGTDY

SEQ ID NO: 139 - protein, 7 aa, artificial sequence
  CDR
GRTFSDY

SEQ ID NO: 140 - protein, 6 aa, artificial sequence
  CDR
NWSGTH

SEQ ID NO: 141 - protein, 11 aa, artificial sequence
  CDR
GWGPQLPGTDY

SEQ ID NO: 142 - protein, 8 aa, artificial sequence
  CDR
GFRFTSYW

SEQ ID NO: 143 - protein, 8 aa, artificial sequence
  CDR
INTGGGST

SEQ ID NO: 144 - protein, 15 aa, artificial sequence
  CDR
ARDLSGSDYVVGITS

SEQ ID NO: 145 - protein, 5 aa, artificial sequence
  CDR
SYWMH

SEQ ID NO: 146 - protein, 17 aa, artificial sequence
  CDR
AINTGGGSTYYADSVKG

SEQ ID NO: 147 - protein, 13 aa, artificial sequence
  CDR
DLSGSDYVVGITS

SEQ ID NO: 148 - protein, 7 aa, artificial sequence
  CDR
GFRFTSY

SEQ ID NO: 149 - protein, 6 aa, artificial sequence
  CDR
NTGGGS

SEQ ID NO: 150 - protein, 13 aa, artificial sequence
  CDR
DLSGSDYVVGITS

SEQ ID NO: 151 - protein, 8 aa, artificial sequence
  CDR
GFSISTYT

SEQ ID NO: 152 - protein, 10 aa, artificial sequence
  CDR
ISPLRWGQST

SEQ ID NO: 153 - protein, 13 aa, artificial sequence
  CDR
CSRPDGKRGQGTQ

SEQ ID NO: 154 - protein, 5 aa, artificial sequence
  CDR
TYTMT

SEQ ID NO: 155 - protein, 20 aa, artificial sequence
  CDR
TISPLRWGQSTTSYADSVKG

SEQ ID NO: 156 - protein, 4 aa, artificial sequence
  CDR
PDGK

SEQ ID NO: 157 - protein, 7 aa, artificial sequence
  CDR
GFSISTY

SEQ ID NO: 158 - protein, 9 aa, artificial sequence
  CDR
SPLRWGQST

SEQ ID NO: 159 - protein, 4 aa, artificial sequence
  CDR
PDGK

SEQ ID NO: 160 - protein, 8 aa, artificial sequence
  CDR
GFTLDYHA

SEQ ID NO: 161 - protein, 8 aa, artificial sequence
  CDR
ISSSGGRT

SEQ ID NO: 162 - protein, 20 aa, artificial sequence
  CDR
AAEIFDSAWYCPLSRYDMDY

SEQ ID NO: 163 - protein, 5 aa, artificial sequence
  CDR
YHAIG

SEQ ID NO: 164 - protein, 17 aa, artificial sequence
  CDR
CISSSGGRTNYADSVKG

SEQ ID NO: 165 - protein, 18 aa, artificial sequence
  CDR
EIFDSAWYCPLSRYDMDY

SEQ ID NO: 166 - protein, 7 aa, artificial sequence
  CDR
GFTLDYH

SEQ ID NO: 167 - protein, 6 aa, artificial sequence
  CDR
SSSGGR

SEQ ID NO: 168 - protein, 18 aa, artificial sequence
  CDR
EIFDSAWYCPLSRYDMDY

SEQ ID NO: 169 - protein, 8 aa, artificial sequence
  CDR
GFTLDYYA

SEQ ID NO: 170 - protein, 8 aa, artificial sequence
  CDR
ISSSGGRT

SEQ ID NO: 171 - protein, 19 aa, artificial sequence
  CDR
AAVILDNSWHCGYSYDMDY

SEQ ID NO: 172 - protein, 5 aa, artificial sequence
  CDR
YYAIG

SEQ ID NO: 173 - protein, 17 aa, artificial sequence
  CDR
CISSSGGRTNYADSVKG

SEQ ID NO: 174 - protein, 17 aa, artificial sequence
  CDR
VILDNSWHCGYSYDMDY

SEQ ID NO: 175 - protein, 7 aa, artificial sequence
  CDR
GFTLDYY

SEQ ID NO: 176 - protein, 6 aa, artificial sequence
  CDR
SSSGGR

SEQ ID NO: 177 - protein, 17 aa, artificial sequence
  CDR
VILDNSWHCGYSYDMDY

SEQ ID NO: 178 - protein, 8 aa, artificial sequence
  CDR
GFTLDYYA

SEQ ID NO: 179 - protein, 8 aa, artificial sequence
  CDR
ISSSGGRS

SEQ ID NO: 180 - protein, 19 aa, artificial sequence
  CDR
AAVILDDSWQCGYYYNMDY

SEQ ID NO: 181 - protein, 5 aa, artificial sequence
  CDR
YYAIG

SEQ ID NO: 182 - protein, 17 aa, artificial sequence
  CDR
CISSSGGRSNYADSVKG

SEQ ID NO: 183 - protein, 17 aa, artificial sequence
  CDR
VILDDSWQCGYYYNMDY

SEQ ID NO: 184 - protein, 7 aa, artificial sequence
  CDR
GFTLDYY

SEQ ID NO: 185 - protein, 6 aa, artificial sequence
  CDR
SSSGGR

SEQ ID NO: 186 - protein, 17 aa, artificial sequence
  CDR
VILDDSWQCGYYYNMDY

SEQ ID NO: 187 - protein, 8 aa, artificial sequence
  CDR
GIMFSYNT

SEQ ID NO: 188 - protein, 7 aa, artificial sequence
  CDR
ITRDGST

SEQ ID NO: 189 - protein, 12 aa, artificial sequence
  CDR
NLGTTDWRRYNY

SEQ ID NO: 190 - protein, 5 aa, artificial sequence
  CDR
YNTMA

SEQ ID NO: 191 - protein, 16 aa, artificial sequence
  CDR
TITRDGSTNYADSMKG

SEQ ID NO: 192 - protein, 10 aa, artificial sequence
  CDR
GTTDWRRYNY

SEQ ID NO: 193 - protein, 7 aa, artificial sequence
  CDR
GIMFSYN

SEQ ID NO: 194 - protein, 5 aa, artificial sequence
  CDR
TRDGS

SEQ ID NO: 195 - protein, 10 aa, artificial sequence
  CDR
GTTDWRRYNY